(19) United States
(12) Patent Application Publication
(10) Pub. No.: US 2015/0159221 A1
(43) Pub. Date: Jun. 11, 2015

(54) CANCER PROGNOSTIC ASSAYS

(71) Applicant: CHILDREN'S HOSPITAL LOS ANGELES, Los Angeles, CA (US)

(72) Inventors: Robert C. Seeger, Los Angeles, CA (US); Shahab Asgharzadeh, Los Angeles, CA (US); Weiyao Liu, Alhambra, CA (US); Richard Sposto, Burbank, CA (US); Lingyun Ji, Los Angeles, CA (US)

(73) Assignee: CHILDREN'S HOSPITAL LOS ANGELES, Los Angeles, CA (US)

(21) Appl. No.: 14/407,046

(22) PCT Filed: Jun. 14, 2013
(86) PCT No.: PCT/US13/45981

§ 371 Date: Dec. 10, 2014

Related U.S. Application Data
(60) Provisional application No. 61/660,584, filed on Jun. 15, 2012.

(51) Int. Cl. C12Q 1/68; G06F 19/00; G06F 19/20

(52) U.S. Cl. CPC C12Q 1/6886; G06F 19/20; G06F 19/3431; C12Q 2600/158; C12Q 2600/16; C12Q 2600/112; G01N 2333/916; G01N 2333/70535; G01N 2333/70596; G01N 2333/99; G01N 2333/912; G01N 2333/70503; G01N 2333/7155; G01N 2333/70567; G01N 2333/4706; G01N 2333/5428; G01N 2333/988; G01N 2333/90245; G01N 2333/4703; G01N 2333/90206; G01N 2333/70539; G01N 2333/70514; G01N 2333/70578; G01N 2333/70532; G01N 2333/70517; G01N 2333/53; G01N 2333/7153; G01N 2333/70521; G01N 2333/7158; G01N 2333/521; G01N 2333/71; G01N 2333/96436; G01N 2333/57; G01N 2333/5437; G01N 2333/5443; G01N 2333/5406; G01N 2333/5412; G01N 2333/5418; G01N 2333/5421; G01N 2333/52; G01N 2333/49

(57) ABSTRACT

The present invention provides for molecular analysis of gene expression of tumors at diagnosis as well as molecular analysis of tumor and normal cell gene expression in bone marrow and blood at diagnosis and during and after completion of treatment. These molecular analyses of gene expression provide an assessment of risk of recurrence and response to therapy of tumors, and in particular, neuroblastoma.

The invention was made with government support under Grant Nos. CA60104 and CA152809-01 awarded by the National Cancer Institute. The government has certain rights to the invention.

### FIELD OF INVENTION

This invention relates to diagnostic and prognostic assays for the assessment of risk of recurrence and response to therapy of a tumor; particularly, a neuroblastoma.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The concept of tumor-promoting inflammation is a recognized enabling characteristic of cancers.¹ Recent studies have demonstrated the prognostic significance of tumor-associated macrophages (TAM) in some adult cancers including Hodgkin's lymphoma and breast cancer.²⁻⁶ However, the prognostic significance of tumor associated inflammatory cells in metastatic disease and in childhood cancers is unknown.

Neuroblastoma, an embryonal tumor of the sympathetic nervous system, is one of the most common solid tumors in children, with approximately 40% of patients presenting with metastatic disease at diagnosis.⁷ Molecular characterization of cancers and improved understanding of tumor biology have led to improvement in treatments with the introduction of targeted therapies, immunotherapy, and combination treatments. Nevertheless, patients still relapse. Early detection of a poor or incomplete response to treatment may allow risk-stratification of patients so that they may receive more effective therapy and thus be less likely to relapse.

Amplification of MYCN (N-myc) protooncogene (30% of tumors) is associated with high risk of disease relapse, while those lacking MYCN amplification (MYCN-nonamplified neuroblastoma (NBL-NA)) have clinical behaviors that are distinctly associated with age at diagnosis.^{8,9} Patients diagnosed with metastatic NBL-NA at ≧18 months of age have tumors with recurrent segmental genomic alterations and have high-risk disease with only 45% long-term, disease-free survival.¹⁰⁻¹⁶ In contrast, children diagnosed <18 months of age have tumors with whole chromosomal alterations and have greater than 90% overall survival after receiving only moderate intensity chemotherapy.^{17,18} Biological mechanisms responsible for the age-dependent genomic and clinical phenotypes of metastatic NBL-NA and for different responses to treatment among those ≧18 months of age at diagnosis have been unclear.

The inventors' previous gene expression profiling study of metastatic NBL-NA tumors suggested that there may be age-dependent differences in expression of genes representing tumor-associated inflammatory cells.¹⁹ Herein, the inventors focused upon intra-tumor inflammatory cells, especially tumor associated macrophages (TAMs), and their relationship to clinical behavior of metastatic NBL-NA. The inventors examined the infiltration of macrophages in loco-regional and metastatic tumors with immunohistochemistry. The inventors assessed expression of inflammatory and tumor cell related genes in metastatic NBL-NA tumors diagnosed before and after 18 months of age. The inventors have developed a highly sensitive and specific molecular assay for detecting and quantifying neuroblastoma cells in blood and bone marrow that provides a new means of assessing response to therapy that is more sensitive than currently available clinical methods. The inventors' findings provide new insights about intra-tumor inflammation in metastatic NBL-NA tumors and provide the basis for constructing a novel 14-gene model that predicts risk of disease progression in those diagnosed ≧18 months of age.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with various embodiments of the invention, evidence of Tumor-associated Macrophages and Inflammation in Neuroblastoma. a) Representative immunohistochemical analyses of staining of CD163 and AIF1 in primary tumor samples from a patient with stage 1 tumor (left panel) lacking any infiltrating macrophages and a patient with metastatic disease (right panel) with extensive infiltration of CD163+ macrophages. b) Average scores for the presence of CD163+ infiltrating macrophages reveals significant infiltration in tumor samples of patients with metastatic disease compared to those with locoregional tumors. Patients with stage 4S tumors, who are known to undergo spontaneous regression, had CD163+ macrophages comparable to those with locoregional tumors (Bonferroni adjusted P<0.017 considered significant). c) Heatmap of gene expression levels of inflammation-related genes displayed along increasing age at diagnosis of 133 children with metastatic NBL-NA. Heatmap colors reflect fold change value of individual tumor relative to the average expression level of inflammation-related genes in children diagnosed <18 months of age. Occurrences of an event (PFS) and of death (OS) are indicated by black vertical bars above the color coded bar for age at diagnosis.

FIG. 2 depicts, in accordance with various embodiments of the invention, Progression-Free Survival (PFS) for Patients in the Training and Validation Cohorts with NBL-NA 14-Gene Signature Low- and High-risk Scores. The cut-off value used to categorize patients into signature-based high- and low-risk score groups depended on the median score obtained in the CCG cohort. The high-risk score group had prediction scores higher than the median score while the low-risk score group had prediction scores lower than the median. The graphs show Kaplan-Meier estimates of PFS for patients with metastatic neuroblastoma lacking MYCN gene amplification according to 14-gene signature risk classification. PFS estimates using the LOOCV signature classification for a) the entire CCG cohort and b) the CCG patients diagnosed at ≧18 months of age (clinically-defined high-risk group, n=94). The classification model developed using the CCG samples was then used to identify signature-based groups for patients treated on c) GPOH high-risk protocols and d) COG high-risk protocols. All P values are based on log rank test.

FIG. 3 depicts, in accordance with various embodiments of the invention, inflammation- and tumor-related Gene-Gene Correlations. a) Heatmap of the Spearman rank correlation matrix of the 14 genes in the NBL-NA signature. Pairwise rank correlation analyses were performed for all 14 genes and age at diagnosis. The patterns of correlation using samples from CCG patients diagnosed ≧18 months of age were similar to the pairwise rank correlations obtained using samples from the GPOH and COG validation cohorts (FIG. 3A). The red color represents positive rank correlation level above zero (white color) for a given gene pair and the blue color represents negative rank correlation level. The inflammation-related genes (FCGR3/CD16, CD33, CD14, IL6-R, IL-10) show high levels of correlation across all cohorts. NTRK2 has the strongest correlation of any tumor cell-related gene with inflammation-related genes. b) Expression of inflammation-related genes is predominantly present in neuroblastoma tumors and not on cell lines. Normalized expression (ACT) values of four of the five inflammation-related genes (CD14, FCGR3/CD16, IL-10, IL-6R) in the CCG tumors (n=133) were compared to six neuroblastoma MYCN non-amplified cell lines (CHLA-15, CHLA-20, CHLA-255, CHLA42, CHLA-90, LAN-6). Data on the cell lines were generated on a TLDA card that did not include CD33 probes. On average, there were 309, 271, 7, and 5-fold higher expression of CD14, FCGR3/CD16, IL-10, and IL-6R in tumors than in cell lines, respectively, suggesting that these genes are primarily expressed by tumor-associated inflammatory cells. c) Scatter diagram of expression of CD14, a macrophage marker, and IL-6R reveals a high correlation in patients with metastatic NBL-NA. d) Similarly, NTRK2, a tumor-cell related gene, shows moderate correlation with expression of IL-6R, an inflammation-related gene. Black=patient diagnosed <18 months of age with metastatic NBL-NA; blue=disease-free patients diagnosed at ≧18 months of age with metastatic NBL-NA, red=disease progression in patients diagnosed ≧18 months of age with metastatic NBL-NA.

FIG. 4 depicts, in accordance with various embodiments of the invention, an overview of the strategy used to develop and validate the NBL-NA Gene Signature. Logistic regression model for each gene and age-at-diagnosis was carried out to identify genes that are predictive of outcome and independent of age at diagnosis. Genes with P≦0.25 were then used in a multi-variate logistic regression model to obtain risk scores for whether progression of disease will or will not occur. Risk groups (low-score or high-score groups) were simply based on the median score of the training set (n=133). This strategy was cross-validated using leave-one-out cross validation. The final model was tested on two independent cohorts of patients.

FIG. 5 depicts, in accordance with various embodiments of the invention, Progression-Free Survival of Children Diagnosed with Metastatic Neuroblastoma Lacking MYCN Amplification by Age at Diagnosis. The graph shows no statistical difference in PFS for the patients clinically identified as high-risk (≧18 months of age at diagnosis) between the training (CCG) and validation (COG, GPOH) cohorts (P value=0.15).

FIG. 6 depicts, in accordance with various embodiments of the invention, the Accuracy of Prediction and Progression-Free Survival based on Re-substitution and LOOCV Analysis in the Training Cohort According to the NBL-NA 14-Gene Signature. The accuracy of the NBL-NA 14-gene signature was obtained using the receiver-operating-characteristic curves (ROC) of this model based on its true positive rate (sensitivity) and false positive rate (1-specificity). The measured Area Under the Curve (AUC) value of the ROC curve using re-substitution and leave-one-out cross-validation (LOOCV) tumor-progression scores demonstrates high accuracy of the signature in A & C) the entire CCG cohort (n=133) and B &D) the CCG patients diagnosed at ≧18 months of age (clinically-defined high-risk group, n=94), respectively. The median re-substitution value of the tumor-progression score of the entire CCG cohort was used to categorize patients into low- (score less than the median value) and high-risk score (score equal or greater than the median value) groups. The re-substitution analysis often over-estimates the accuracy of a given model and this bias reflects the need for cross-validation.

FIG. 7 depicts, in accordance with various embodiments of the invention, Progression-Free Survival based on Re-substitution Analysis in the Training Cohort According to the NBL-NA 14-Gene Signature. The graphs show PFS estimates using the 14-gene signature classification scores based on re-substitution analysis for A) the entire CCG cohort and B) the CCG patients diagnosed at ≧18 months of age (clinically-defined high-risk group, n=94). The re-substitution analysis often over-estimates the accuracy of a given model and this bias reflects the need for cross-validation.

FIG. 8 depicts, in accordance with various embodiments of the invention, (A) Predicted Five-year Cure Rate vs. Percent of Samples Grouped as Low Risk (all CHLA patients) Note: Lines were fit using loess smoothing function [1]; (B) Predicted Five-year Cure Rate vs. Percent of Samples Grouped as Low Risk (CHLA patients>=18 months).

FIG. 9 depicts, in accordance with various embodiments of the invention, (A) LOOCV—Predicted Five-year Cure Rate vs. Percent of Samples Grouped as Low Risk (All CHLA Patients); (B) LOOCV—Predicted Five-year Cure Rate vs. Percent of Samples Grouped as Low Risk (CHLA Patients>=18 Months).

FIG. 10 depicts, in accordance with various embodiments of the invention, (A) External Validation—EFS for German patients>=18 months (Cutoff point: median predicted probability of failure for all CHLA patients); (B) External validation—Predicted Five-year Cure Rate vs. Percent of Samples Grouped as Low Risk (German Patients>=18 Months).

FIG. 11 depicts, in accordance with various embodiments of the invention, between detection score and immunocytology. The immunocytology (IC) analysis of bone marrow samples performed at the COG reference laboratory detects the number of tumor cells per 1 million mononuclear cells by microscopically counting tumor cells after staining with a cocktail of 4 neuroblastoma specific antibodies (x-axis). The NBL-Detect assay data (y-axis) was obtained from RNA extracted from the same aliquot used to generate the IC data. The figure shows high correlation between the two assays when tumor cells are detected by IC. However, a significant number of samples have NBL-Detect signal (blue circles) despite lack of NB cell detection IC. Almost, all samples that have undetectable signal in NBL-Detect (green circles) have no evidence of NB cells by IC (open circles; extremely low False Negative rate).

FIG. 12 depicts, in accordance with various embodiments of the invention, correlation between “Tumor Load” in Day 1 PBSC and Event-Free Survival—COG-A3973.

FIG. 13 depicts, in accordance with various embodiments of the invention, correlation between “Tumor Load” in Day 1 PBSC and Overall Survival—COG-A3973.

FIG. 14 depicts, in accordance with various embodiments of the invention, increasing “Tumor Load” in Day 1 PBSC Correlates with Worse Event-Free Survival—COG-A3973.

FIG. 15 depicts, in accordance with various embodiments of the invention, increasing “Tumor Load” in Day 1 PBSC Correlates with Worse Overall Survival—COG-A3973.

FIG. 16 depicts, in accordance with various embodiments of the invention, increasing “Tumor Load” in Day 1 PBSC Correlates with Worse EFS—COG-A3973.

FIG. 17 depicts, in accordance with various embodiments of the invention, event-free survival in relationship to tumor content of bone marrow at 3 and 9 months after ABMT.

FIG. 18 depicts, in accordance with various embodiments of the invention, overall survival in relationship to tumor content of bone marrow at 3 and 9 months after ABMT.

FIG. 19 depicts, in accordance with various embodiments of the invention, event-free survival in relationship to tumor content of bone marrow at 3 months after ABMT.

FIG. 20 depicts, in accordance with various embodiments of the invention, overall survival in relationship to tumor content of bone marrow at 3 months after ABMT.

FIG. 21 depicts, in accordance with various embodiments of the invention, event-free survival in relationship to tumor content of bone marrow at 9 months after ABMT.

FIG. 22 depicts, in accordance with various embodiments of the invention, overall survival in relationship to tumor content of bone marrow at 9 months after ABMT.

FIG. 23 depicts, in accordance with various embodiments of the invention, a schema for utilization of TLDA based assays and other clinical parameters by the clinical trial researchers. Clinical performance characteristics are assessed at defined decision points that would allow clinicians to alter treatment course for children with ultra-high risk of relapse.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

The diverse outcomes of children with metastatic neuroblastoma lacking genomic amplification of the MYCN proto-oncogene (NBL-NA; MYCN non-amplified, neuroblastoma) have been largely unexplained. The inventors' study suggests for the first time that infiltrating inflammatory cells, especially TAMs, may contribute to this diversity. The inventors demonstrate that TAMs are more prevalent in tumors of children with metastatic than loco-regional neuroblastoma. Further, the inventors show that expression of inflammation-related genes is higher in tumors of children diagnosed ≧18 month of age, and that a subset of these genes representing TAMs is associated with an extremely poor outcome in this group. Including expression of both inflammatory and tumor cell genes in a 14-gene signature enables prediction of disease progression for the first time in the clinically indistinguishable group of patients diagnosed ≧18 month of age with metastatic NBL-NA. The novel finding that five inflammation-related genes contribute to 25% of the accuracy of the 14-gene model emphasizes the role of inflammation in neuroblastoma and uncovers previously unrecognized potential targets for therapy. This 14-gene expression scoring model, which was validated in two independent cohorts of patients, has clinical applicability and may be of used for managing high-risk patients.

Genes related to tumor cells contributed most to the accuracy of the 14-gene NBL-NA signature and included NTRK2, which binds brain-derived neurotrophic factor and plays an important role in the survival and differentiation of neuroblastomacells.^{27,28,39,49} The association of high NTRK2 expression with aggressive behavior in metastatic NBL-NA was previously reported by the inventors' group¹⁹, and is further supported in this study. The inventors' present work also reveals a novel association between expression of IL-6R and NTRK2. The inventors' data point to the role of a pro-tumor inflammatory microenvironment in enabling a highly aggressive neuroblastoma phenotype.

Several gene expression and genomic studies of neuroblastoma tumors obtained at diagnosis have previously reported associations with patient outcomes.⁴¹⁻⁴⁶ However, these studies analyzed groups of patients who were heterogeneous with respect to MYCN gene amplification status, clinical stage, and age at diagnosis. Segmental genomic alterations identified in high-risk NBL-NA tumors have not been predictive of outcome in this high-risk group, but have clinical utility in children with intermediate-risk neuroblastoma.^{43,44,47-51} Gene expression-based models built using neuroblastoma samples from clinically heterogeneous groups of patients lack predictive accuracy in those diagnosed ≧18 months of age with metastatic NBL-NA.^{45,46} Similarly, the inventors' previously reported 55-gene NBL-NA specific microarray signature¹⁹ was not predictive of outcome for patients with MYCN amplified tumors, which represent a distinct molecular subgroup of neuroblastomas (unpublished data and ⁵²). Overall, these findings suggest that prognostic studies in neuroblastomas should focus on well-defined molecular and clinical subgroups (e.g. using MYCN status). The inventors' current finding also highlights the importance of assessing the neuroblastoma tumor microenvironment in prognostic studies.

The invention described herein defines a clinically applicable 14-gene expression signature (NBL-prognostic; NBL-prog) that identifies two subsets of patients with different progression-free survival (PFS). Children with high-risk tumor progression scores uniformly had a poor outcome with 8% to 20% PFS at five years after diagnosis, while those with low-risk scores had 47% to 57% PFS. Addition of treatment response evaluations such as imaging with ¹²³Imetaiodobenzylguanidine and quantification of bone marrow disease with PCR assays may further improve risk classification, especially for patients with a low-risk tumor progression score.^{60,61}

The inventors' study reports the first evidence of a role for intra-tumor inflammation in metastatic neuroblastomas and provides a validated prognostic signature for children with metastatic NBL-NA. The increase in expression of inflammation-related genes in children≧18 months of age with poor outcome allows the identification of a subgroup of patients at extremely high-risk who may benefit from treatments targeting the tumor microenvironment along with tumor cells. The recent success of therapies directed at tumor-associated immune system cells in adult cancers⁶²⁻⁶⁴ suggests opportunities for their application in children with neuroblastoma.

Embodiments of the present invention provide a highly sensitive and specific method for quantifying circulating and bone marrow tumor cells that may serve as a surrogate for clinical response and as an early warning of impending relapse in patients with cancer.

Embodiments of the present invention provide a new and nonobvious processes, systems and compositions to detect tumor cells in bone marrow and blood in neuroblastoma patients (NBL-detect). The NB detection score is the geometric mean of cycle threshold (CT) values of the set of 5 detection genes.

A TaqMan™ Low-Density-Array assay was created that uses a panel of 44 genes chosen for their ability to both identify neuroblastoma cells and characterize the microenvironment of normal cells surrounding the tumor cells. The assay quantifies neuroblastoma cells in blood and bone marrow with 5 genes that are strongly expressed by neuroblastoma but rarely by normal cells. This provides highly sensitive quantification of “tumor load” in blood and bone marrow (1 tumor cell in 10⁶ normal cells can be detected) and provides prognostic information. The assay also quantifies expression of 39 genes by normal cells that may affect tumor cell growth and survival. This test may be particularly useful in evaluating treatments and predicting outcome in children and, with modifications, adults with cancer.

Embodiments of the present invention also provide a novel method for recovering high quality RNA from viable fresh and frozen specimens for use in the test.

Embodiments of the present invention provide for a diagnostic tool to evaluate treatment response. It can be applied to other types of cancer than neuroblastoma with modification of genes tested. The advantages of the test are 1) specificity for tumor cells; 2) high sensitivity; 3) provides information about normal cells in bone marrow that may impact tumor cell growth and response to treatment; 4) allows determining blood contamination in bone marrow samples; and 5) provides high quality RNA extraction from frozen samples

The inventors developed a TaqMan® Low Density Array (TLDA) assay that quantifies expression of five genes (chromogranin A (CHGA), doublecortin (DCX), dopadecarboxylase (DDC), paired-like homeobox 2B (PHOX2B), and tyrosine hydroxylase (TH) that are highly expressed by NBL cell lines and tumors and are rarely expressed by normal blood cells. This assay has a 6-log dynamic range and a detection sensitivity of one tumor cell per million normal cells. Data are reported as positive or negative for tumor cells and as the geometric mean Cycle Threshold (Ct) of the expression of the five genes (detection gene score=DG). It was found that tumor is more often detectable in BM than in blood, and the DG is higher in BM, although the 2 are correlated.

Further, accurate quantification of tumor burden in neuroblastoma patients is needed to define homogenous populations for therapy and establish response criteria that predict outcome. The 5-gene TLDA assay was developed for sensitive quantification of neuroblastoma cells in bone marrow (BM) and blood. This TLDA assay detects neuroblastoma cells in both BM and blood in patients with recurrent/refractory neuroblastoma at high rates, and it frequently detects tumor cells when BM morphology and imaging evaluations do not.

Additionally, assessing the response of neuroblastoma cells in bone marrow (BM) to therapy with a highly sensitive and quantitative assay may provide warning of relapse. The TaqMan® Low Density Array (TLDA) platform was used to 1) identify five genes that are strongly expressed by neuroblastoma but not by normal hematopoietic cells; 2) compare TLDA and immunocytology assays for quantifying tumor cells in bone marrow; and 3) assess the ability of the TLDA assay to predict event-free survival (EFS). This 5-gene TLDA assay provides a sensitive and quantitative test for neuroblastoma cells in bone marrow that identifies patients at high-risk for disease progression.

Various embodiments of the present invention are based, at least in part, on the above findings, which are further detailed in the embodiments and the examples below.

Various embodiments provide for a process, comprising providing a first composition comprising a plurality of isolated nucleic acids probes; contacting the first composition to an RNA sample from a mammalian subject desiring a determination of the likelihood of neuroblastoma recurrence or response to chemotherapy, to produce one or more cDNA molecules; providing a second composition comprising one or more isolated nucleic acids probes comprising a sequence capable of hybridizing to one or more nucleic acids selected from the group of genes consisting of PTPN5, GPATC4, H2AFV, FCGR3A; FCGR3B, CD14, PGM2L1, NTRK2, CD33, THAP2, IL6R, GFRA3, CAMTA1, IL10, and BTBD3; contacting the second composition with the one or more cDNA molecules to amplify the one or more cDNA molecules; and quantifying the expression level of the one or more genes to determine the likelihood of neuroblastoma recurrence or response to chemotherapy in the mammalian subject. In various embodiments, the RNA sample is obtained from a tumor sample.

In various embodiments, the process further comprises determining a progression score. The progression score can be determined by the following formula:

$$B + {\sum }_{i = 1}^{14} {{c}_{i} ( Δ CT gene )}_{i}$$

wherein B is the intercept value, c is the coefficient of a gene, i is index of summation, and ΔCT is the change in cycle threshold value for the gene.

In various embodiments, the coefficients can be found in Table 10. In various embodiments, the intercept value is −3.38.

The progression score can be used to determine a course of therapy. For example, identification of the most aggressive tumors by the presence of tumor associated macrophages (TAMs) within them could lead to improved chemotherapy treatments in the first six months after diagnosis if the pro-tumor interactions between tumor cells and TAMs were blocked. Pre-clinical research indicates that drugs such as lenalidomide and sorafenib block these interactions and significantly improves the response of tumors in mice to standard chemotherapy drugs cyclophosphamide and topotecan. Thus, in various embodiments, lenalidomide or sorafenib can be used in a course of therapy. Furthermore, these interactions also result in immune suppression, and blocking them significantly improves the anti-tumor activity of Natural Killer (NK) cells combined with anti-tumor antibodies in mouse tumor models. Improved immunotherapy would be important for elimination of minimal residual disease after chemotherapy has been completed.

Various embodiments of the present invention provide for a process for determining the likelihood of neuroblastoma recurrence or response to chemotherapy, in a subject in need thereof, comprising: providing a sample from the subject; determining an expression level of genes selected from the group consisting of: PTPN5, GPATC4, H2AFV, FCGR3A; FCGR3B, CD14, PGM2L1, NTRK2, CD33, THAP2, IL6R, GFRA3, CAMTA1, IL10, and BTBD3, of the sample; determining a progression score for the subject, wherein a progression score of above a median risk score indicates a high likelihood of neuroblastoma recurrence or low likelihood of response to chemotherapy, a progression score below a median risk score indicates a low likelihood of neuroblastoma recurrence or a high likelihood of response to chemotherapy. In various embodiments, the median risk score is 0.68. This particular median risk score was calculated using the training cohort of patients (CCG, n=133 patients). Children whose tumors have risk score below this median value were considered to have low progression scores and those with tumor risk scores above the median value have high progression scores.

In various embodiments, the subject is a child with MYCN non-amplified metastatic neuroblastoma. In various embodiments, the progression score is determined by the formula as indicated above. In various embodiments, the coefficient is also from Table 10. In various embodiments, intercept value is −3.38. Again, the progression score can be used to determine a course of therapy.

Various embodiments of the present invention provides for a process to detect a tumor cell, comprising: providing a first composition comprising a plurality of isolated nucleic acids probes; contacting the first composition to an RNA sample from a mammalian subject desiring a diagnosis or prognosis regarding a tumor (e.g., brain tumor, neuroblastoma) to produce one or more cDNA molecules; providing a second composition comprising isolated nucleic acids probes comprising a sequence capable of hybridizing to nucleic acids selected from the group of detection genes consisting of chromogranin A (“CHGA”), doublecortin (“DCX”), dopadecarboxylase (DDC), paired-like homeobox 2B (“PHOX2B”), and tyrosine hydroxylase (“TH”); contacting the second composition with the one or more cDNA molecules to amplify the one or more cDNA molecules; and quantifying the expression level of the detection genes to detect a tumor cell. In various embodiments, the process further comprises determining a detection gene score from the expression level of the detection genes. A detection gene score 40 or higher is an indication of an absence of a tumor cell and a low likelihood of disease progression. A detection gene score between 37 and 40 indicates a presence of a tumor cell and a medium likelihood of disease progression. A detection gene score that is less than 37 indicates the presence of a tumor cell and a high likelihood of disease progression.

In various embodiments, the second composition further comprises one or more isolated nucleic acids probes comprising a sequence capable of hybridizing to one or more nucleic acids selected from the group of housekeeping genes consisting of beta-2 microglobulin (“B2M”), glyceraldehyde-3-phosphate dehydrogenase (“GAPDH”), hypoxanthine guanine phosphoribosyl transferase (“HPRT1”), succinate dehydrogenase complex, subunit A (“SDHA”); and the process further comprises quantifying the expression level of the one or more housekeeping genes.

In various embodiments, the second composition further comprises one or more isolated nucleic acids probes comprising a sequence capable of hybridizing to one or more nucleic acids selected from the group of microenvironment genes consisting of CD14, CD16 (FCGR3B; 3A), CD163, CD19, CD34, CD4, CD40LG, CD86, CD8A, CSF1 (M-CSF), CSF1R (CD115), CTLA4, CX3CR, CXCL12, CXCR3, CXCR4, FLT1 (VEGFR1), FOXP3, GNLY, GZMB, HMOX1, IFNG, IL10, IL13, IL15, IL2RA, IL4, IL6, IL6R, IL7, IL7R, IL8, KDR (VEGFR2), KLRK1 (NKG2D), NCAM1, TBX21, TEK, TGFB1, and VEGFA; and the process further comprises quantifying the expression level of the one or more microenvironment genes. The expression level of the one or more microenvironment genes provides information regarding the quality of a bone marrow sample with regard to its contamination by blood cells, which dilute the bone marrow cells and hence render detection of tumor cell less sensitive. For example, expression of the 39 microenvironment genes allow identification of blood vs. bone marrow in that CD4 and CD8 expression is higher in blood and CD34 is higher in bone marrow

In various embodiments, the RNA sample is obtained from mononuclear cells, bone marrow cells, blood, or peripheral blood stem cell (“PBSC”).

Various embodiments of the present invention also provides a composition, comprising: one or more isolated nucleic acids probes comprising sequences capable of hybridizing to the group of detection genes consisting of chromogranin A (“CHGA”), doublecortin (“DCX”), dopadecarboxylase (DDC), paired-like homeobox 2B (“PHOX2B”), and tyrosine hydroxylase (“TH”). In various embodiments, the composition also comprises one or more isolated nucleic acids probes comprising a sequence capable of hybridizing to one or more nucleic acids selected from the group of housekeeping genes consisting of beta-2 microglobulin (“B2M”), glyceraldehyde-3-phosphate dehydrogenase (“GAPDH”), hypoxanthine guanine phosphoribosyltransferase (“HPRT1”), succinate dehydrogenase complex, subunit A (“SDHA”). In various embodiments, the composition further comprises one or more isolated nucleic acids probes comprising a sequence capable of hybridizing to one or more nucleic acids selected from the group of microenvironment genes consisting of CD14, CD16 (FCGR3B; 3A), CD163, CD19, CD34, CD4, CD40LG, CD86, CD8A, CSF1 (M-CSF), CSF1R (CD115), CTLA4, CX3CR, CXCL12, CXCR3, CXCR4, FLT1 (VEGFR1), FOXP3, GNLY, GZMB, HMOX1, IFNG, IL10, IL13, IL15, IL2RA, IL4, IL6, IL6R, IL7, IL7R, IL8, KDR (VEGFR2), KLRK1 (NKG2D), NCAM1, TBX21, TEK, TGFB1, and VEGFA. In various embodiments, the composition further comprises: one or more isolated cDNA molecules transcribed from an RNA sample obtained from a mammalian subject desiring a diagnosis or prognosis regarding a tumor.

Various embodiments of the present invention also provide for an array, comprising a substrate having any of the above-described compositions.

Various embodiments of the present invention also provide for a process of selecting a therapy for a patient, comprising: providing a subject expression profile of a biological sample from the patient; providing a plurality of reference profiles, each associated with a therapy, wherein the subject expression profile and each reference profile has a plurality of values, each value representing a detection gene score determined from the expression level of detection genes selected from the group consisting of chromogranin A (“CHGA”), doublecortin (“DCX”), dopadecarboxylase (DDC), paired-like homeobox 2B (“PHOX2B”), and tyrosine hydroxylase (“TH”); and selecting the reference profile most similar to the subject expression profile, to thereby select a therapy for said patient. For example, a patient who is found to be responding poorly to initial standard chemotherapy could receive an alternative therapy that may more effectively attack both tumor cells and tumor promoting microenvironment cells in the bone marrow or tumor tissue. A patient responding poorly to immunotherapy would similarly receive alternative immunotherapy that may be more effective by suppressing the microenvironment while enhancing the immune response. In various embodiments, the process further comprises administering the selected therapy.

Various embodiments provide for a process of diagnosing or prognosticating a tumor (e.g., brain tumor, neuroblastoma), comprising: providing one or more probes to detect the expression of one or more genes from a group of detection genes consisting of chromogranin A (“CHGA”), doublecortin (“DCX”), dopadecarboxylase (DDC), paired-like homeobox 2B (“PHOX2B”), and tyrosine hydroxylase (“TH”); contacting the one or more probes test sample obtained from a mammalian subject desiring a diagnosis or prognosis regarding the tumor; determining one or more expression levels of the one or more detection genes; diagnosing or prognosticating the tumor based on the one or more expression levels.

In various embodiments, the process further comprises determining the detection gene score. In various embodiments, a determination of the absence of a tumor cell is made when the detection gene score is 40 or higher and it is indicative of a low likelihood of disease progression. In various embodiments, a determination of the presence of a tumor cell is made when the detection gene score is between 37 and 40 and it is indicative of a medium likelihood of disease progression. In various embodiments, a determination the presence of a tumor cell is made when the detection gene score is less than 37 and it is indicative of a high likelihood of disease progression.

Various embodiments of the present invention provide for a process of prognosticating a tumor of a mammalian subject, comprising: obtaining a progression score and a detection gene score; combining the progression score with the detection gene score; and determining a risk profile based on the combination of the progression score and the detection gene score For example, approximately 40% of patients with a “good” tumor progression score nevertheless develop progressive disease and die. Evaluating bone marrow and blood tumor cell responses to their therapy using the detection gene score will reveal those who are poor responders to therapy. This provides the opportunity to change therapy for these patients and possibly improve their outcome.

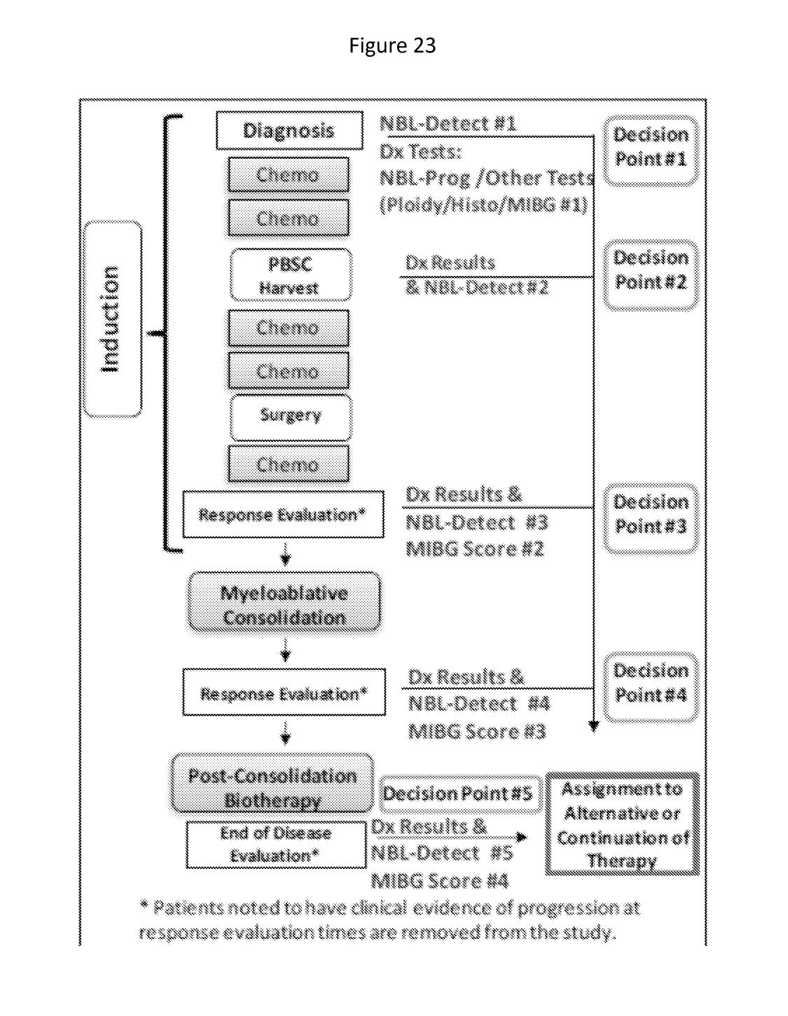

Combined assessment of progression score and available pathobiology markers for primary tumors at diagnosis (e.g., degree of tumor differentiation, Mitosis Karyorrhexis Index [MKI], and genomic ploidy) plus measurement of tumor load and response to therapy in bone marrow, PBSC, and/or blood at defined evaluation times by detection score (e.g. FIG. 23 Decision Point 2 or 3) in combination with the MIBG imaging Curie score may provide the most accurate identification of subgroups at risk of neuroblastoma recurrence (FIG. 23).

Statistical analysis is used to determine if combining the TLDA assays (NBL-Prog (progression scores) and NBL-Detect (detection scores)) by themselves or in combination with other biologic and imaging studies provides the most accurate prediction of outcome. This evaluation utilizes centrally reviewed DNA ploidy and histological data including tumor differentiation status and mitosis-karyorrhexis index (MKI). TLDA assays increase the sensitivity of identifying children at ultra-high risk of disease recurrence while maintaining a low false positive rate (children who will be mistakenly identified as ultra high-risk). Thus analyses assesses if a clinically meaningful increase in sensitivity (greater than 10% over existing models) can be achieved at a false positive rate of 20%. Addition of progression scores to models that include histology and ploidy are evaluated at diagnosis. Addition of detection score from bone marrow, blood, and PBSC at diagnosis, early in induction, (Decision Point #2 in FIG. 23), end of induction (Decision Point #3 in FIG. 23), end of consolidation (Decision Point #4 in FIG. 23), and end of therapy (Decision Point #5 in FIG. 23) are compared to a model including only radiolabeled metaiodobenzylguanidine (MIBG) scoring. Additionally, we will evaluate these models by including at each decision point, the amount of change in NBL-Detect from its immediate previous decision point. Two approaches to analyze these multivariate models will be used to determine whether combining data sets significantly improves prediction of outcome compared to the individual evaluations.

Inventors provide a clinically relevant and feasible combination of prognostic factors with which to identify a cohort of high risk patients for whom conventional high risk therapy is very unlikely to be curative, e.g., “ultra-high risk” (UHR). We hypothesize that the progression score, detection scores, and/or MIBG Curie score, will provide prognostic information from diagnosis. Because detection scores and MIBG score are available at multiple time points during frontline therapy, this gives us the opportunity to test their utility and prognostic strength at multiple Decision Points (FIG. 23). In clinical practice, one or two, Decision Points are selected at which to shunt the UHR patients to alternative novel therapies.

Two analytic approaches are undertaken: A) Survival tree regression of PFS, using a univariate Cox proportional hazards regression model, as per Cohn et al. Factors tested include binary factors age, presence of metastases, ploidy, MKI, and grade of differentiation. From statistically significant factors, the factor with the maximum hazard ratio is selected to create a given split/branch; this process is repeated within each branch/node. Based on the long-term PFS of the tree's terminal nodes, patients are classified as either standard high-risk (SHR) or ultra-high risk (UHR). To obtain bias-corrected Kaplan-Meier (KM) estimates of the long-term EFS probabilities for each group (SHR and UHR), a 0.632 bootstrap cross validation is employed (49, 52). This entire survival tree building process is repeated from scratch, with the addition of NBL-Prog (binary), NBL-Detect (binary), and MIBG score (binary) (where appropriate for a given Decision Point) as potential prognostic factors to be tested. The binary variables for NBL-Prog and NBL-Detect are created using the cut points identified by the analysis of progression and detection scores. The binary form of MIBG Curie score will use a cut-off value of 2, per Naranjo et al. The cross-validated KM estimates of PFS from the two models is compared to see if there is substantial improvement in correctly identifying UHR patients. In the second model, a logistic regression modeling of long-term PFS as in Asgharzadeh et al. is used. Whether to perform logistic-regression modeling of a landmark indicator of long-term PFS or logistic regression in the context of a parametric cure model depends on the follow-up maturity of the sample.

The advantage of the survival tree regression approach is sensitive to possible complex interactions between covariates, such as the observation that ploidy is probably a more important prognostic factor in younger children than older ones. It has the disadvantage that it is more difficult to adjust the classification to target different levels of sensitivity and specificity. The main-effects logistic regression approach, in contrast, will not capture profound quantitative or qualitative interactions. However, this method produces a continuous prognostic score (e.g., the predicted probability of long-term PFS), which can be used to construct ROC curves and to adjust the cut points to achieve different levels of sensitivity and specificity.

Various embodiments provide for a process of processing a frozen sample from a mammalian subject for obtaining RNA from the frozen sample, comprising: providing a quantity of a RNA stabilization reagent; contacting the quantity of the RNA stabilization reagent with the frozen sample until the frozen sample is thawed, thereby producing a first mixture; centrifuging the first mixture, thereby producing a first supernatant and pelleted cells; remove the first supernatant; place a quantity of a RNA isolation reagent comprising phenol and guanidine isothiocyanate in contact with the pelleted cells, thereby producing a second mixture; lyse the cells by pipetting the second mixture; transfer the lysed cells to a microtube; incubate the lysed cells for a first period of time at approximately room temperature; contact a quantity of chloroform to the lysed cells, thereby producing a third mixture; mix the third mixture; allow the third mixture to stand at room temperature a second period of time; centrifuge the third mixture at about 4° C., thereby producing a mixture comprising an upper aqueous phase; transfer the upper aqueous phase to a microfuge tube; contact a quantity of isopropyl alcohol to the upper aqueous phase, thereby producing a fourth mixture; mix the fourth mixture; let the fourth mixture stand at room temperature for a third period of time; centrifuge the fourth mixture for about 20 minutes at about 4° C., thereby producing a fourth mixture comprising a supernatant and a precipitate; remove the supernatant; contact a quantity of cold ethanol to the precipitate, thereby producing a fifth mixture; centrifuge for about 2 minutes at about 4° C., thereby producing a fifth mixture comprising a supernatant and a precipitate; remove the supernatant; allow the precipitate to dry for about 3 minutes; contact a quantity of RNase-free water to the precipitate, thereby producing a sixth mixture, vortex and spin the sixth mixture; contacting a quantity of a lysis buffer containing 1% 0-Mercaptoethanol to the sixth mixture, thereby producing a seventh mixture; mix and spin the seventh mixture; contact a quantity of ethanol to the seventh mixture, thereby producing an eighth mixture; and pipet and transfer the eighth mixture immediately to spin column (e.g., RNeasy spin column). In various embodiments, the sixth mixture can be stored at about −80° C. until further use.

Examples of “brain tumors” that are diagnosed or prognosticated in accordance with various embodiments of the present invention include but are not limited to neuroblastomas, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, and craniopharyngiomas.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Macrophages were identified using immunohistochemical (IHC) analysis of primary neuroblastoma tissues using antibodies directed against CD163 and AIF1 (allograft inflammatory factor 1). Tissue sections scores ranged from 0 to 7 for each marker, with higher scores indicating a greater proportion of positive cells.

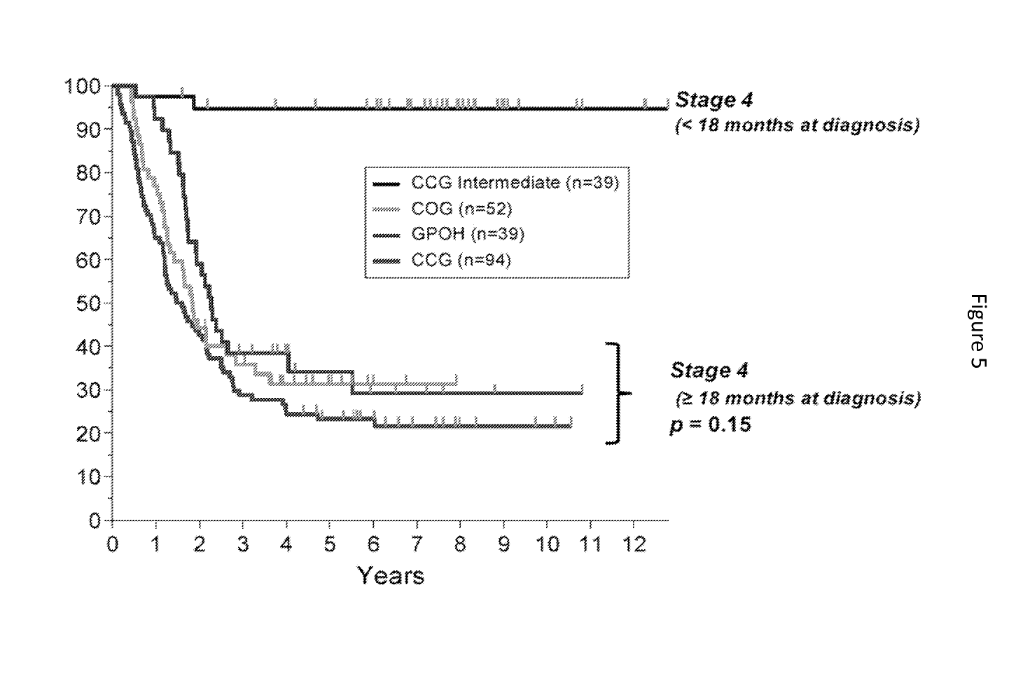

The details of the 48-gene TLDA assay are provided in Table 2, and the examples below. All patients included in the gene expression study had metastatic NBL-NA tumors and were enrolled in Children's Cancer Group (CCG), German Society for Pediatric Oncology-Hematology (GPOH), or COG trials at diagnosis (Table 1; FIG. 5). Details of treatment for the three cohorts were described previously and provided in the examples belo.^{11,14,16,19} Informed consent was obtained in accordance with institutional review board policies.

TABLE 1
Characteristics of patients with metastatic neuroblastoma lacking
MYCN gene amplification*

| | Characteristic | | | |
|---|---|---|---|---|
| | Training Cohort | | Validation Cohorts | |
| | CCG | | GPOH | COG |
| | Age at diagnosis | | | |
| | <18 months | ≧18 months | ≧18 months | ≧18 months |
| | (n = 39) | (n = 94) | (n = 39) | (n = 52) |
| Mean age at diagnosis in | 9.3 | 53.6 | 56 | 55.9 |
| months (range) | (0.1-17.3) | (18.2-151) | (18.4-182) | (19.5-186) |
| COG Risk-Stratification | Intermediate^{†} | High | High | High |
| INPC Classification - no. (%) | | | | |
| Favorable | 34 (87) | 2 (2) | 3 (8) | 3 (6) |
| Unfavorable | 5 (13) | 91 (97) | 34 (87) | 42 (81) |
| Unknown | 0 | 1 (1) | 2 (5) | 7 (13) |
| Clinical Trials | 323P, 3881 | 323P, 321-2, | NB90, NB95, | A3973^{††} |
| | | 321-3, 3891 | NB97, NB2004 | |
| 5-year EFS Rate (95% CI) | 95% | 23% | 34% | 31% |
| | (81%, 99%) | (15%, 32%) | (19%, 50%) | (19%, 44%) |
| 5-year OS Rate (95% CI) | 95% | 35% | 50% | 38% |
| | (81%, 99%) | (25%, 44%) | (32%, 66%) | (23%, 54%) |

*CCG = Children's Cancer Group; GPOH = German Society for Pediatric Oncology and Hematology (Gesellschaftfur Paediatrische Onkologie und Haematologie); COG = Children's Oncology Group; INPC = International Neuroblastoma Pathology Classification.
^{†}Patients older than 12 months of age were classified as high-risk and treated accordingly per CCG guidelines. Based on current COG guidelines, 4 out of the 12 patients diagnosed at 12-18 months of age with unfavorable tumor histology would be considered high-risk.
^{††}Three patients were treated on ANBL0532

TABLE 2
List of tested genes and their univariate significance of association and predictive
accuracy of event, after adjusting for continuous age.

| | | | P Value from | | AUC for |
|---|---|---|---|---|---|
| | | | Likelihood Ratio | AUC for Training | Training cohort |
| Rank | ABI Probe Name | Gene Symbol | Test | cohort | >=18 mo at diagnosis |
| 1 | Hs00370894_m1 | H2AFV | 0.0001 | 0.8308 | 0.6275 |
| 2 | Hs00737786_g1 | GPATC4 | 0.0003 | 0.8262 | 0.6060 |
| 3 | Hs00262161_s1 | PTPN5 | 0.0006 | 0.8133 | 0.5636 |
| 4 | Hs00328100_m1 | PGM2L1 | 0.0017 | 0.8061 | 0.5127 |
| 5 | Hs01063366_m1 | GNAI1 | 0.0018 | 0.8117 | 0.4971 |
| 6 | Hs00186495_m1 | TMEFF1 | 0.003 | 0.8147 | 0.5268 |
| 7 | Hs00415042_m1 | IGKC; IGKV1 | 0.0048 | 0.8145 | 0.5486 |
| 8 | Hs00275547_m1 | FCGR3A; FCGR3B | 0.0058 | 0.8156 | 0.5649 |
| 9 | Hs00326433_m1 | NXPH1 | 0.0212 | 0.8205 | 0.5225 |
| 10 | Hs00275009_s1 | CNR1 | 0.023 | 0.8062 | 0.4814 |
| 11 | Hs00854282_g1 | C5orf13 | 0.0265 | 0.8090 | 0.5258 |
| 12 | Hs00171267_m1 | TGFB1 | 0.0283 | 0.8051 | 0.6290 |
| 13 | Hs00173925_m1 | GPR86 | 0.036 | 0.8087 | 0.4873 |
| 14 | Hs00233544_m1 | CD33 | 0.0392 | 0.8048 | 0.5179 |
| 15 | Hs00171690_m1 | HOXC6 | 0.0487 | 0.8099 | 0.4905 |
| 16 | Hs00181751_m1 | GFRA3 | 0.0501 | 0.8044 | 0.4729 |
| 17 | Hs00230167_m1 | THAP2 | 0.0542 | 0.7993 | 0.4703 |
| 18 | Hs00176787_m1 | NTRK1 | 0.0612 | 0.7931 | 0.4442 |
| 19 | Hs00705034_s1 | SMARCE1 | 0.0617 | 0.8011 | 0.4814 |
| 20 | Hs00174131_m1 | IL6 | 0.0631 | 0.7966 | 0.4808 |
| 21 | Hs00209118_m1 | BTBD3 | 0.0634 | 0.7936 | 0.4514 |
| 22 | Hs00930455_m1 | CXCL12 | 0.0672 | 0.8023 | 0.5016 |
| 23 | Hs00169842_m1 | IL6R | 0.083 | 0.8002 | 0.4977 |
| 24 | Hs00391998_m1 | CAMTA1 | 0.1034 | 0.7956 | 0.4494 |
| 25 | Hs00383314_m1 | C6orf168 | 0.1406 | 0.7998 | 0.4644 |
| 26 | Hs02621496_s1 | CD14 | 0.1498 | 0.8028 | 0.5036 |
| 27 | Hs00299139_s1 | YPEL1 | 0.201 | 0.8011 | 0.4664 |
| 28 | Hs01016341_m1 | ST7 | 0.2157 | 0.8016 | 0.4690 |
| 29 | Hs00174086_m1 | IL10 | 0.2193 | 0.7991 | 0.4814 |
| 30 | Hs02786786_s1 | NTRK2 | 0.2338 | 0.8025 | 0.4886 |
| 31 | Hs00644818_m1 | MS4A1 | 0.2883 | 0.7899 | 0.4553 |
| 32 | Hs00392922_g1 | MYT1L | 0.2918 | 0.7920 | 0.4827 |
| 33 | Hs00225656_m1 | C1orf35 | 0.2941 | 0.7968 | 0.4710 |
| 34 | Hs00220252_m1 | PARP6 | 0.3671 | 0.7982 | 0.4599 |
| 35 | Hs00234174_m1 | STAT3 | 0.3716 | 0.7966 | 0.4853 |
| 36 | Hs00174333_m1 | CD19 | 0.3819 | 0.7908 | 0.4534 |
| 37 | Hs00227602_m1 | PRG2 | 0.3949 | 0.8030 | 0.4684 |
| 38 | Hs00705213_s1 | HRK | 0.4326 | 0.7938 | 0.4449 |
| 39 | Hs00379318_m1 | PAK7 | 0.4394 | 0.7968 | 0.4508 |
| 40 | Hs00365842_m1 | CX3CR1 | 0.7708 | 0.7979 | 0.4579 |
| 41 | Hs00194072_m1 | APBA2 | 0.8091 | 0.7972 | 0.4547 |
| 42 | Hs00289942_s1 | LOC284244 | 0.85 | 0.7975 | 0.4540 |
| 43 | Hs00268388_s1 | SOX4 | 0.9115 | 0.7968 | 0.4534 |
| 44 | Hs00366902_m1 | SCN3A | 0.9456 | 0.7966 | 0.4547 |

*To develop a clinically-applicable prognostic assay for patients diagnosed ≧18 months of age with metastatic NBL-NA, the inventors used the TaqMan ® Low Density Array (TLDA) to obtain qRT-PCR gene expression values of 44 genes in 133 samples from the CCG training cohort. Genes were selected based on available TLDA probes matching the genes identified in the inventors' previously published 55-gene microarray signature (24 matching genes with mean spearman correlation of r = 0.61SD ± 0.2), previous studies in neuroblastoma, and inflammation-related genes selected based on prior knowledge and identification by gene set enrichment analysis of neuroblastoma microarray data. Thirty-two of the 44 (73%) genes related to tumor cells, while 12 of the 44 (23%) were inflammation-related genes.

### Example 2

### Statistical Analysis—Metastatic NBL-NA Signature

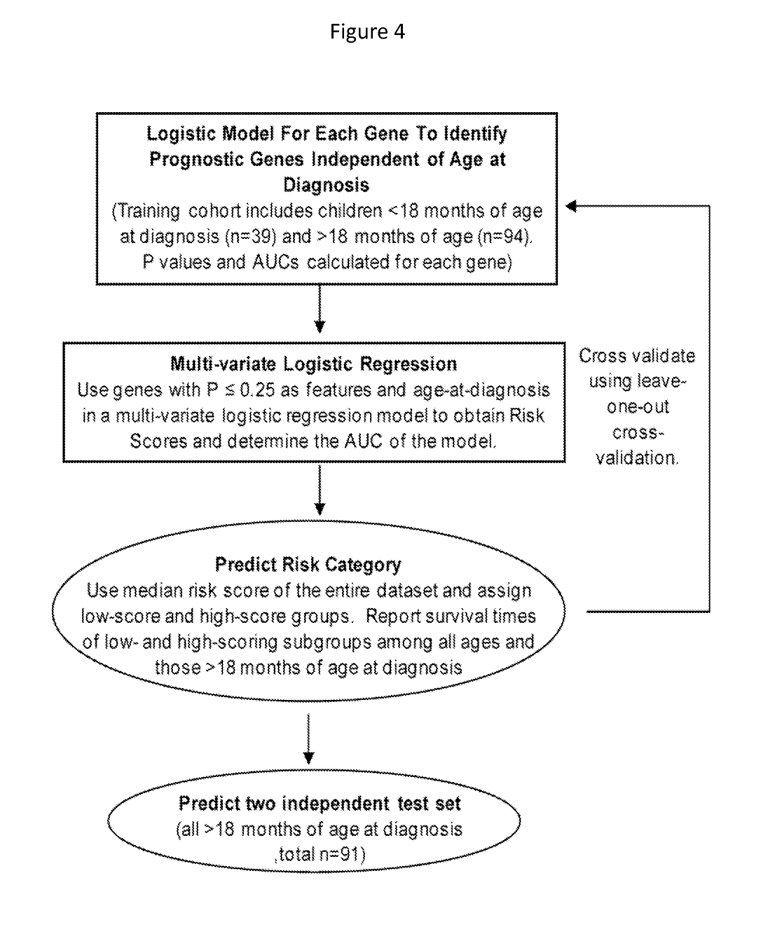

FIG. 4 illustrates the flow of statistical and validation methods used herein. As a primary interest was to identify genes that are predictive of outcome in the cohort of patients older than 18 months of age, the inventors first used a univariate logistic regression model based on TLDA gene expression data from 133 samples from the training cohort (CCG), which includes patients older and younger than 18 months of age at the time of diagnosis. Genes that were independent of age at diagnosis with a P value of ≦0.25 were included in a final multivariate logistic model to predict PFS. The inventors' aim was to build a robust model that was predictive of disease progression in patients older than 18 months of age and that could be used as the basis for classification into signature-based low- and high-risk tumor-progression groups. Disease progression was defined a priori. The effective period for risk of disease progression in the training cohort was 4 years from diagnosis. Because few patients were censored before the end of the effective period for risk of disease progression, ignoring this censoring had little practical effect on the logistic regression analysis of whether or not disease progression had occurred. Age was included as a continuous covariate in the final multivariate logistic regression analysis to assess for residual significance. The logit values, representing the tumor-progression scores, were computed for each patient. Measures of accuracy based on re-substitution analysis and leave-one-out cross-validation (LOOCV) are presented. Classification accuracy was assessed using receiver-operating-characteristic (ROC) curves and areas-under-the-curve (AUC). External validation of the prediction model was performed using the independent GPOH and COG samples, with the tumor-progression score for each patient calculated using the regression coefficients from the prediction model derived from the training cohort. The relative contributions to the accuracy of the 14-gene NBL-NA prediction score of age at diagnosis, tumor cell-related genes, and inflammation-related genes were assessed using 5000 permutations of the dataset

Tumor-progression risk scores obtained from the multivariate logistic regression model were used to define signature-based risk groups. The median tumor-progression risk score from the training cohort (n=133) was used as the cut-off point to define signature-based high-risk (tumor-progression score≧median score) or low-risk (tumor-progression score<median score) scores.

### Example 3

### Statistical Methods

Details of the statistical analyses for developing the prognostic score are described above and summarized in FIG. 4. In addition, survival analysis methods²² are used to describe outcome in low- and high-risk groups defined by the prognostic score. The primary endpoint for these analyses was progression-free survival (PFS), defined as the minimum interval from date of diagnosis to date of disease progression, date of death (4 patients only), or date of last follow-up. Patients who did not progress or expire were censored at the time of the last follow-up. The Kaplan-Meier method was used to compute PFS probabilities and produce survival curves. Confidence intervals are based on Greenwood standard errors. Unless otherwise stated, the reported probabilities are based on 5-year PFS rates. Tests of the difference in PFS between risk groups are based on the log-rank statistic. Other common statistics²³ (e.g., Student's t-test, Spearman rank correlation) are used where appropriate and are indicated in the text. Bonferroni adjustments to account for multiple comparisons are used where appropriate. Statistical computations were performed using STATA software (version-9.0; StataCorp, TX) or the R project.

### Example 4

### Infiltration of Inflammatory Cells in Metastatic Neuroblastoma

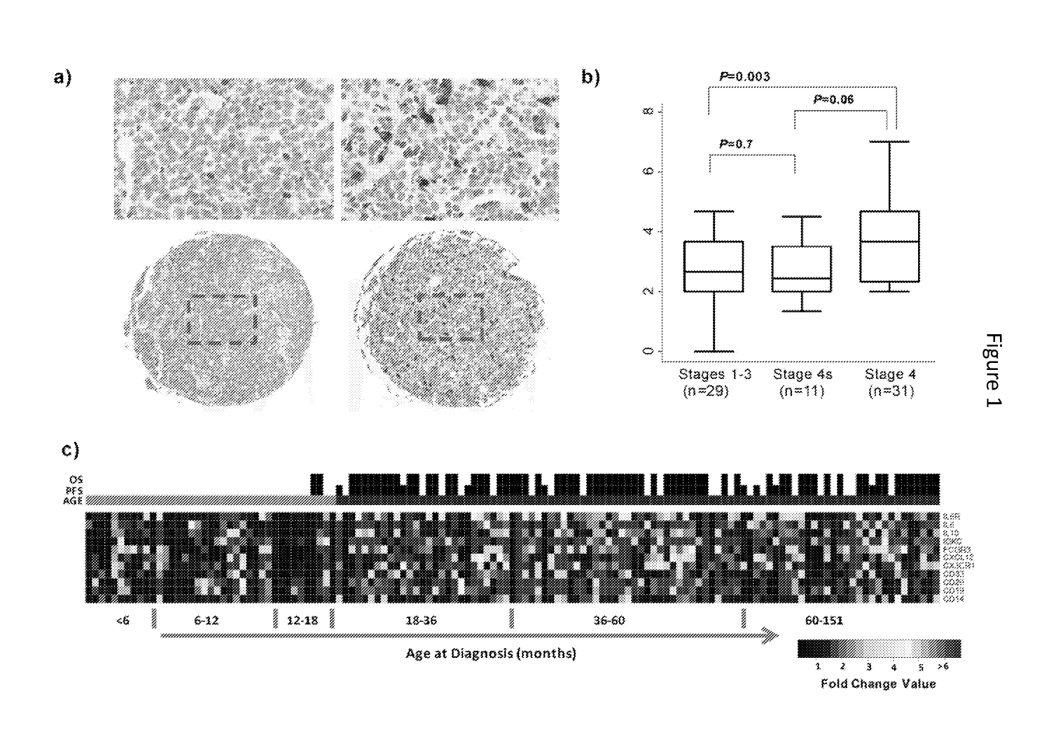

The inventors performed immunohistochemical analysis of 71 neuroblastoma tumors (29 patients with loco-regional, 31 with metastatic disease [stage 4], and 11 with metastatic disease with special designation [stage 4S], Table 3) using antibodies directed against two macrophage markers (CD163 and AIF3). There were significantly greater numbers of infiltrating macrophages observed only with CD163 staining, which identifies alternatively activated macrophage (M2), in samples of patients with metastatic (stage 4) compared to loco-regional neuroblastoma (P=0.003, Student t-test, Bonferroni adjusted P<0.017 considered significant; FIG. 1). There was no statistically significant difference in the number of intra-tumor CD163+ macrophages between metastatic tumors with special designation (stage 4S), which undergo spontaneous regression, and loco-regional tumors.

The inventors also performed gene expression analysis of 133 metastatic NBL-NA tumors (CCG cohort: 94 children diagnosed ≧18 months of age and 39 diagnosed <18 months of age, Table 1) with a custom built TLDA containing 31 tumor-related and 13 inflammation-related genes (Table 2). The inventors identified greater expression of inflammation-related genes associated with monocyte/macrophage, myeloid and B cells in tumors of children diagnosed ≧18 months of age compared to those diagnosed <18 months of age (FIG. 1). Although inflammation-related genes CD33, FCGR3 (CD16), and IGKC showed significant association with progression-free survival (PFS) in univariate analysis (Table 2), the inventors did not identify any single gene model that could accurately predict PFS in children diagnosed ≧18 months of age with AUC>0.7. These data suggest that inflammatory cells within tumors, especially TAMs, contribute to the age-associated clinical behavior of metastatic NBL-NA tumors.

TABLE 3
Characteristics of patients used in IHC analysis*

Characteristic
Age at diagnosis

| | <18 months | ≧18 months |
|---|---|---|
| | (n = 38) | (n = 33) |
| Locoregional | | |
| Stage 1 | 8 | 2 |
| Stage 2 | 9 | 2 |
| Stage 3 | 3 | 5 |
| Metastatic (Stage 4) | | |
| MYCN non-amplified | 5 | 7 |
| MYCN amplified | 1 | 2 |
| missing MYCN status | 1 | 15 |
| Stage 4S | 11 | 0 |

*Analysis was conducted using tissue microarray developed at COG and its Biopathology center as well as cases obtained at Children's Hospital Los Angeles. TMA cores were evaluated by pathologist (HS) to ensure adequate tumor cell content. Ganglioneuromas and ganglioneuroblastomas present on the TMA were excluded from the analyses.

### Example 5

### Expression of Inflammation- and Tumor Cell-Related Genes Comprise a Prognostic Signature

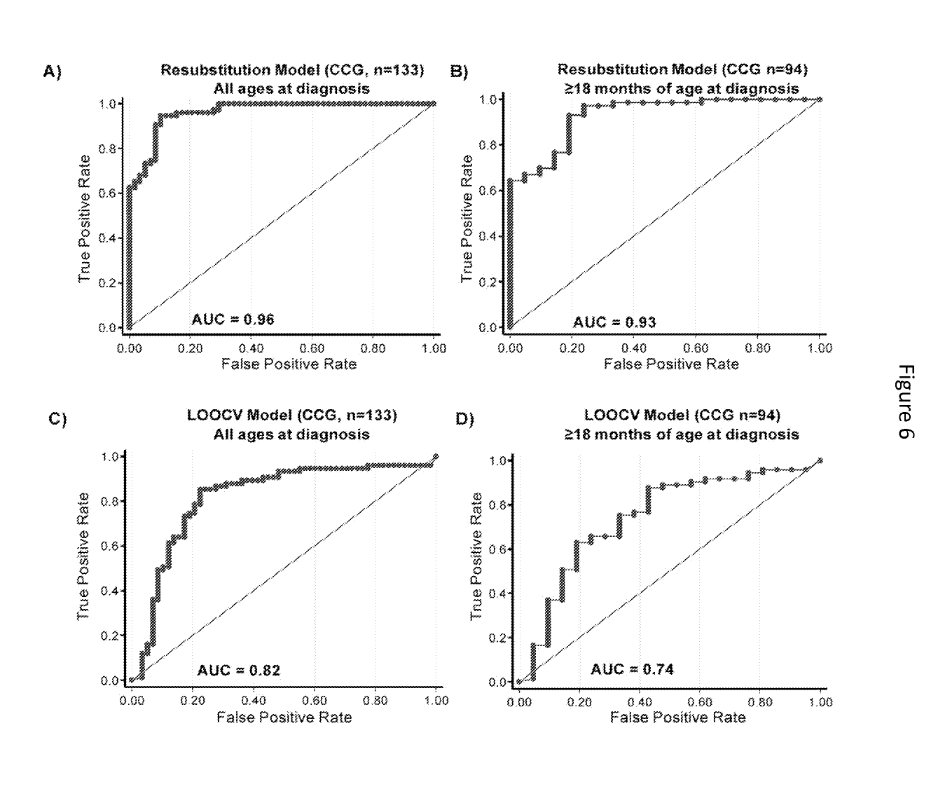

The inventors further examined the expression of the 31 tumor-related and 13 inflammation-related genes in the CCG cohort and identified 14 genes that contributed to a model predictive of progression-free survival (PFS) (Table 4). Among the 14 genes used in the inventors' model, nine (64%) were tumor cell-related and five (36%) were inflammation-related. The accuracy of the model for predicting PFS using leave-one-out-cross-validation (LOOCV) AUC estimates was 0.82 for patients in all age groups and 0.74 for patients≧18 months at diagnosis (FIG. 6).

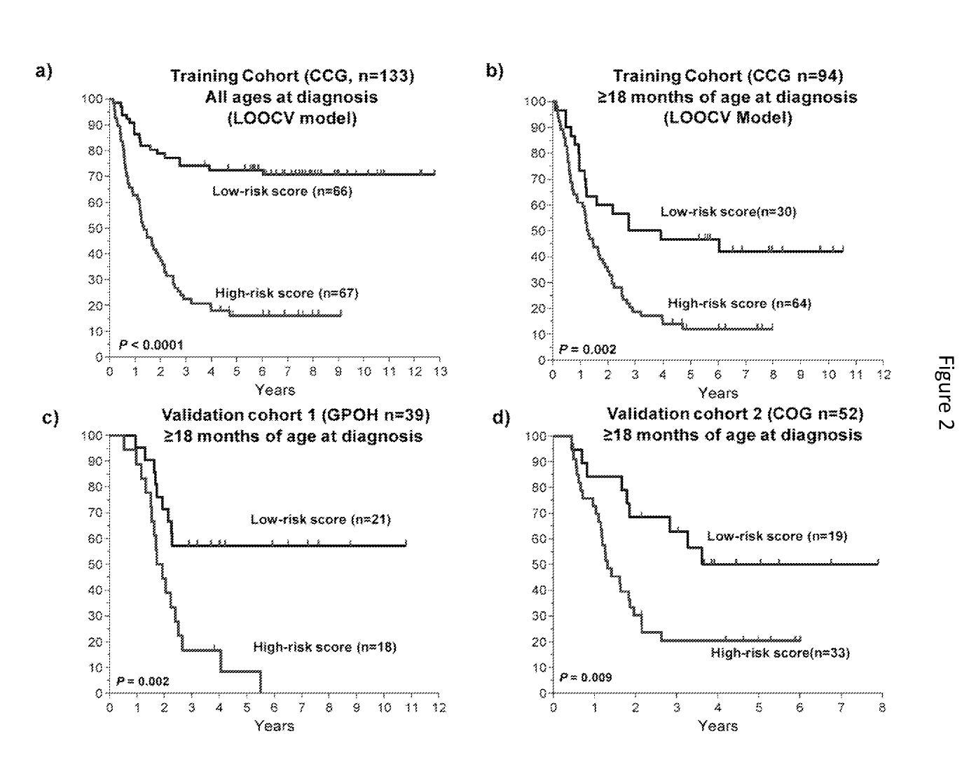

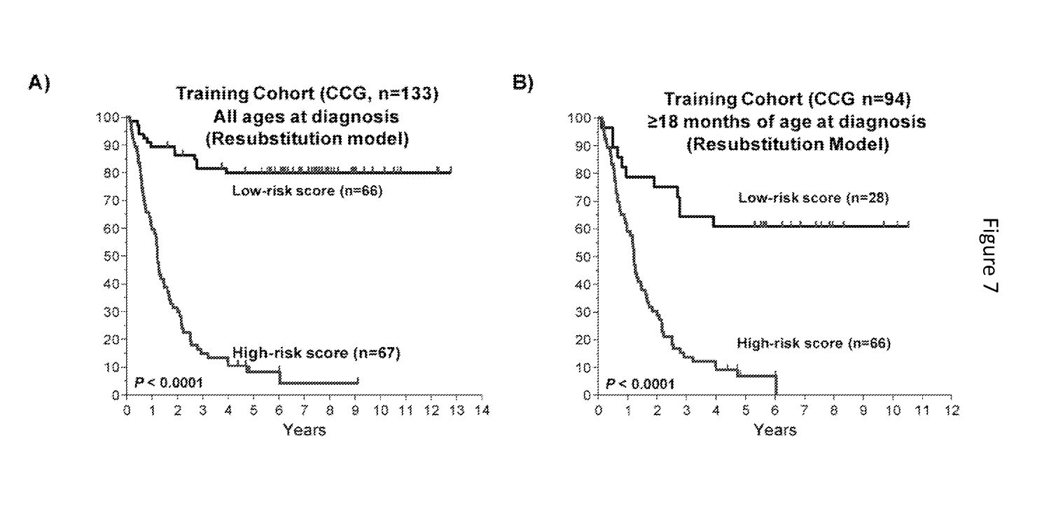

Tumors from the CCG cohort were categorized as low- or high-risk based upon their 14-gene tumor-progression risk score using LOOCV analysis. FIG. 2 shows that patients with a low-risk score had significantly better PFS (72% at 5-years, 95% confidence interval [CI]=60%-82%) than those in the high-risk score group (16% at 5-years, 95% CI=8%-26%) using LOOCV analysis (P<0.0001). The overall 5-year PFS for the 94 patients who were >18 months at diagnosis and treated on CCG high-risk protocols was 23%. Among these patients, 30 (32%) had a low-risk score with a 5-year PFS rate of 47% (95% CI=28%-63%), and 64 (68%) had a high-risk score with a 5-year PFS of 12% (95% CI=5%-22%) (P=0.002), demonstrating that a subset of patients at extremely high-risk of disease progression can be identified by the 14-gene signature among these otherwise clinically indistinguishable patients. Five-year overall survival for the 94 patients whose tumors had low- or high-risk scores also was significantly different (60% vs. 23%, P=0.003; FIG. 2). Classification by re-substitution analysis showed higher accuracy in prediction and more divergent Kaplan-Meier curves (FIGS. 6-7), reflecting the optimistically biased approach of this analysis and the need for cross-validation.²⁶⁻³⁰

Independent validation of the 14-gene signature to predict PFS was obtained from analysis of metastatic NBL-NA tumors from two independent cohorts of patients (GPOH n=39 and COG n=52) diagnosed ≧18 months of age, Table 1 and FIG. 5). Those patients whose tumors had signature-based high-risk scores had significantly worse 5-year PFS than those with low-risk scores (FIG. 2). Among the 39 GPOH patients, 21 (54%) had low-risk scores with a 5-year PFS rate of 57% (95% CI=34%-75%) and 18 (46%) had high-risk scores with a 5-year PFS of 8% (95% CI=1%-29%; P=0.002). Nineteen (36%) COG patients had low-risk scores with a 5-year PFS rate of 50% (95% CI=26%-70%), and 33 (64%) had high-risk scores with a 5-year PFS of 20% (95% CI=8%-35%; P=0.009). Five-year overall survival (OS) for patients in these two cohorts whose tumors had low- or high-risk scores also was significantly different (FIG. 2; GPOH 65% vs. 31%, P=0.012; COG 51% vs. 31%, P=0.039). These data show the validity of the 14-gene signature and the prognostic information obtained from inclusion of inflammation-related genes to identify subsets of patients with different outcomes in a clinically indistinguishable population.

TABLE 4
Characteristics and prognostic significance of the 14 genes of the neuroblastoma signature.

| | | | | | |
|---|---|---|---|---|---|
| | | | | | Prediction |
| | | Gene | Univarite | | Accuracy (AUC)^{††} |
| Symbol | Gene Name | Location | Odds Ratio (95% CI)^{†} | P value | (CCG patients ≧18 mos.) |

Tumor-Related Genes:

| | | | | | |
|---|---|---|---|---|---|
| H2AFV | H2A histone family, member V | 7p13 | 0.42 (0.26, 0.68) | 0.0001 | 0.6275 |
| GPATC4 | G patch domain containing 4 | 1q22 | 2.08 (1.33, 3.24) | 0.0003 | 0.6060 |
| PTPN5 | Protein tyrosine phosphatase, non-receptor type 5 | 11p15.1 | 1.28 (1.10, 1.48) | 0.0006 | 0.5636 |
| PGM2L1 | Phosphoglucomutase 2-like 1 | 11q13.4 | 0.62 (0.45, 0.85) | 0.002 | 0.5127 |
| GFRA3 | GDNF family receptor alpha 3 | 5q31.1 | 0.79 (0.62, 1.01) | 0.05 | 0.4729 |
| THAP2 | THAP domain containing, apoptosis associated | 12q21.1 | 0.59 (0.34, 1.03) | 0.05 | 0.4703 |
| | protein 2 | | | | |
| BTBD3 | BTB (POZ) domain containing 3 | 20p12.2 | 0.76 (0.56, 1.02) | 0.06 | 0.4514 |
| CAMTA1 | Calmodulin binding transcription activator 1 | 1p36.31 | 0.80 (0.60, 1.05) | 0.1 | 0.4494 |
| NTRK2 | Neurotrophic tyrosine kinase, receptor, type 2 | 9q22.1 | 1.16 (0.91, 1.48) | 0.2 | 0.4886 |

Inflammation-Related Genes:

| | | | | | |
|---|---|---|---|---|---|
| FCGR3 | Fc fragment of IgG. CD16 | 1q23 | 1.36 (1.08, 1.72) | 0.006 | 0.5649 |
| (CD16) | | | | | |
| IL-6R | Interleukin 6 receptor | 1q21 | 1.26 (0.97, 1.65) | 0.08 | 0.4977 |
| CD33 | CD33 Antigen | 19q13.3 | 1.34 (1.00, 1.80) | 0.04 | 0.5179 |
| IL-10 | Interleukin 10 | 1q31-q32 | 1.17 (0.91, 1.50) | 0.2 | 0.4814 |
| CD14 | CD14 Antigen | 5q31.1 | 1.25 (0.92, 1.70) | 0.15 | 0.5036 |

^{†}Univariate odds ratio for each gene after adjusting for age at diagnosis for the training CCG cohort (n = 133). Coefficients were calculated based on a two-fold increase in gene expression(i.e. equivalent to a change of 1 ΔCT).
^{††}Area under the curve (AUC) values are reported for the patients diagnosed at ≧18 months of age in the training cohort. A logistic regression model that included the individual gene expression value plus age at diagnosis as a continuous variable was fit to the training cohort (n = 133). The logit scores were used in ROC analysis to obtain AUC values.

### Example 6

### Prognostic Contribution of Genes Related to Tissue-Associated Macrophages

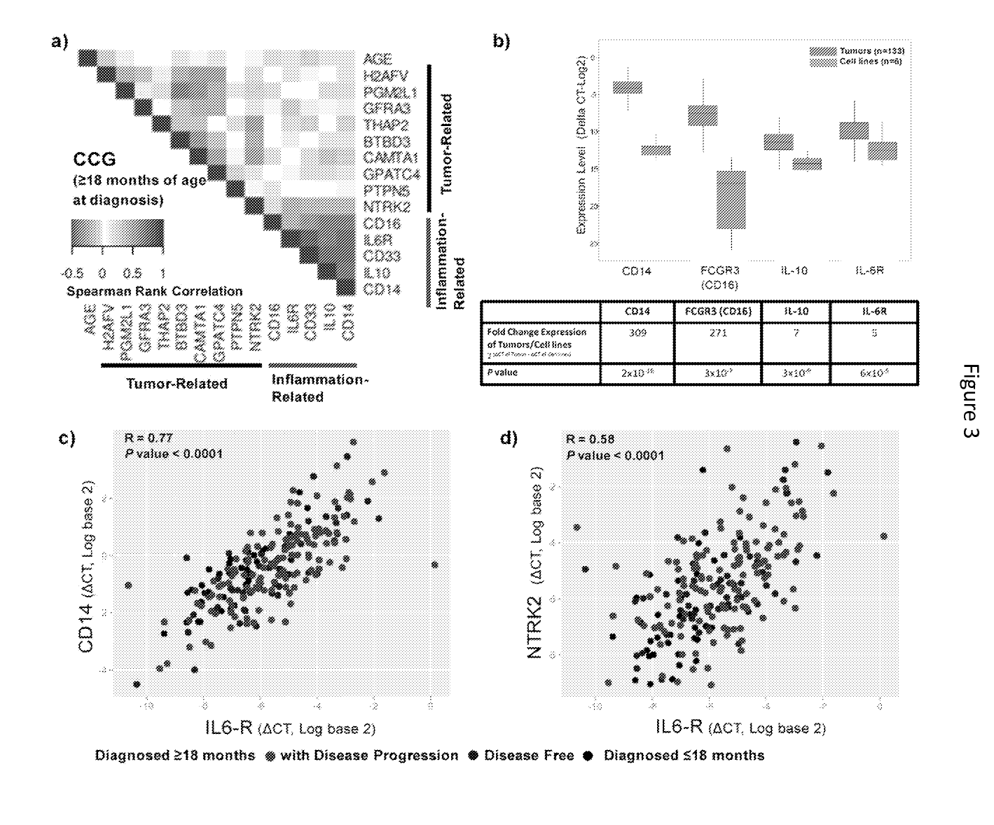

The five inflammation-related genes in our 14-gene model included CD14, CD33, FCGR3 (CD16), interleukin-6 receptor (IL-6R), and interleukin-10 (IL-10), which are mainly expressed by macrophages and myeloid cells and along with CD163 signify intra-tumor macrophage polarization to the anti-inflammatory M2-like phenotype.^{30,31} The inventors' previous research demonstrated that expression of these inflammation-related genes in neuroblastoma tumors correlates with microscopic presence of IL-6-producing CD68+ cells, which are considered to be tumor-associated macrophages.³² Comparison of levels of expression of inflammation-related genes in neuroblastoma tumors to five neuroblastoma cell lines demonstrated that these markers are expressed 150-fold (range: 5-309; P values<0.001, Student t-test) higher on average in tumors than in cell lines (FIG. 3), suggesting that these genes are primarily expressed by tumor-associated inflammatory cells and consistent with IHC finding.

The inventors next investigated the contribution of gene categories and age at diagnosis to the predictive accuracy of the NBL-NA signature. Using a permutation strategy, the inventors discovered that on average the inclusion of inflammation-related genes explained 25% of the accuracy of the 14-gene model in predicting PFS, and added to the 63% provided by tumor-cell related genes. Age at diagnosis explained an additional 12% of the accuracy.

Inflammation-related genes were also found to be highly correlated to each other in the CCG cohort, a pattern that was also observed in the GPOH and COG cohorts (FIG. 3). The strongest gene-gene correlations were observed between IL-6R and CD14 (Spearman r=0.77, P<0.001; FIG. 3), and IL-6R and CD33 (Spearman r=0.75, P<0.001). IL-6R is primarily expressed on cells of the monocytic lineage, but it has also been reported to be expressed on some neuroblastoma cell lines.³³

The nine genes categorized as tumor cell-related included neurotrophic kinase receptor 2 (NTRK2) and calmodulin-binding-transcription-activator-1 (CAMTA1), genes known to be associated with neuroblastoma growth and suppression, respectively.²⁶⁻²⁹ IL-6R was also found to be moderately correlated to the expression of the NTRK2 (Spearman r=0.58; P<0.001; FIG. 3). Together, these data suggest that inflammation-related genes, especially genes related to polarization of TAMs, contribute to prognosis and are associated with a poor clinical outcome.

### Example 7

### Immunohistochemical Studies

Neuroblastoma tissue microarray (TMA) was obtained from the Children's Oncology Group's biopathology center (Columbus, Ohio) and sample tissues from patients treated at Children's Hospital Los Angeles (n=12). The Biopathology TMA contains 90 unique cases (0.6 mm cores in duplicates, 60% of cases or triplicates 40% of cases) with 16 Stage 1, 16 Stage 2, 15 Stage 3, and 29 Stage 4, 15 Stage 4S cases, and twenty control cores (from 3 ganglioneuromas and 3 tonsils). The samples were all stained at ChildLab (a Division of Nationwide Children's Hospital, Columbus, Ohio) based on a double staining procedure and using antibodies against CD163 (Biocare Medical) and AIF1 (Leica Microsystems) with appropriate negative controls and counterstains. All stained sections were scored by three reviewers (HS, MH, SA). Twenty eight TMA cores were not evaluable due to poor tissue preservation, post-treatment samples, minimal presence of adequate neuroblasts, or extensive necrosis/fibrosis. Additional staining was performed on 12 tumor tissues from Children's Hospital Los Angeles. In total, there were 71 samples included in the final analyses: stages 1-3 (n=29), stage 4S (n=11), and metastatic, stage 4 (n=31); see Table 3. Samples with large discrepancies in scores (difference in scores between 2 reviewers greater than 2) were re-reviewed by all three reviewers to reach consensus. Each antibody was scored independently with scores ranging from 0 to 3 allowing increments of 0.5. The scoring system was proposed by HS and reflects percentage of macrophages that occupy the septal spaces between tumor cells (0 <25%, 1: 25-50%, 2: 50-75%, 3: >75%). All scores were re-scaled to integers (0-7) for subsequent analyses.

### Example 8

### Gene Expression Studies

Similar to the inventors' previous strategy for identifying a microarray-based gene expression signature for classification NBL-NA subgroup′, while not wishing to be bound by any particular theory, the inventors believe that the gene expression profiles of tumors from patients younger than 18 months of age when diagnosed with metastatic NBL-NA, who have generally excellent outcomes, could be used to help identify the least aggressive tumors in patients who were 18 months or older at diagnosis. Therapy for patients in the CCG cohort was based on risk categories defined by age at diagnosis. In CCG trials, patients diagnosed before 12 months of age (CCG intermediate-risk group) received multi-agent chemotherapy. Ninety-seven of the 133 samples used in this study were from CCG patients that were also studied in our previous gene expression profiling study. All CCG patients diagnosed after 12 months of age were classified as high-risk according to CCG-3891 clinical trial and received multi-agent induction chemotherapy followed in most cases by randomized assignment to consolidation with either conventional dose chemotherapy or with myeloablative chemoradiotherapy and autologous hematopoietic stem cell transplantation (AHSCT). The high-risk group from CCG-3891 was further randomized to receive 13 cis-retinoic acid or no further therapy. Note that current COG classification has expanded the criteria for classifying neuroblastoma patients at intermediate-risk for disease progression to include all patients who are younger than 18 months at diagnosis and have a tumor that is hyperdiploid with favorable histology according to the International Neuroblastoma Pathology Classification (INPC). This change was the result of reanalysis of the prognostic importance of age at diagnosis, which indicated that any cut point for age at diagnosis between 15 and 19 months was statistically significantly associated with good prognosis among patients diagnosed with metastatic neuroblastoma without MYCN gene amplification.

The majority of GPOH patients and all COG patients received myeloablative chemoradiotherapy consolidation supported with AHSCT. Patients enrolled in COG-A3973 either non-randomly received 13-cis-retinoic acid or were randomized to receive 13-cis-retinoic acid with or without anti-GD2 antibody immunotherapy after myeloablative chemoradiotherapy. All samples were evaluated histologically to confirm diagnosis and features used in the INPC such as assessment of neuroblastic differentiation, mitosis-karyorrhexis index (MKI) and assignment to favorable or unfavorable groups.

Disease progression was defined a priori as the development of any new lesion, a greater than 25% increase in the mass of any measurable tumor, or a previously negative bone marrow sample that became positive for tumor cells. Two patients in the CCG group had inadequate documentation for evidence of relapse and presumed to have a non-disease related event, and two other patients had evidence of disease progression at autopsy. These patients were considered to have disease progression in our analyses.

Total RNA from frozen tumor sections of the CCG samples was previously isolated for microarray analysis using TRIzol reagent at Children's Hospital Los Angeles. TRIzol-based RNA extraction of samples from the COG and GPOH cohorts was conducted at the Children's Oncology Group's Biopathology Center (Columbus, Ohio) or University of Cologne (Cologne, Germany), respectively. RNA quality for all samples was assessed at Children's Hospital Los Angeles by gel electrophoresis and RNA integrity number (RIN) using a Bioanalyzer 2100 (Agilent Technologies, Santa Clara, Calif.). Only the samples with RIN>6.4 were subjected to reverse transcription into cDNA using M-MLV reverse transcriptase with oligo-dT priming (Invitrogen, Carlsbad, Calif.). Quantitative reverse transcription polymerase chain reaction (qRT-PCR) was carried out on a custom-designed TaqMan® Low Density Array (TLDA), using an ABI 7900 Sequence Detection System (Applied Biosystems, Carlsbad, Calif.). Five hundred nanograms of sample cDNA were loaded into the TLDA ports per manufacturer's protocol.

### Example 9

### TLDA Analysis and Gene Selection

The TLDA was constructed with genes related to tumor- and inflammation-related genes. Tumor-related genes were selected based on previously published microarray studies.¹⁻³ Inflammation-related genes were selected based on prior knowledge and gene set enrichment analysis of microarray data.¹ After performing qRT-PCR reactions by TLDA, the cycle threshold (CT) values for the 44 genes with detectible expression (CT<40) in more than 95% of specimens were determined and normalized to the three housekeeping genes (GAPDH/SDHA/HPRT1; Table 2). Cycle threshold (CT) value for each gene was determined as follows: (1) Raw fluorescence values for each PCR cycle were exported from the Applied Biosystems 7900HTVersion 2.3 Sequence Detection Systems software; (2) For each gene within each sample, a baseline value was computed as the median fluorescence from cycles 3-15. To avoid overestimating the baseline for some high-expressing genes, the upper limit of this range was adjusted to a value that was a least 3 cycles lower than the computed CT value for the gene; (3) the baseline value was subtracted from the raw fluorescence values, and a LOESS smoothing function was fitted. A CT value was computed as the point where the smoothed function intersected a fixed threshold value of 0.20. The assay was considered negative if the baseline-corrected function did not intersect the fixed threshold. In these cases, the CT was assigned the value 40 (this occurred in less than 0.3% of all reactions).

Preliminary analysis of data generated using a 96-gene TLDA array and 40 tumor samples from the CCG cohort led to a 48-gene TLDA assay that was then used with the training and validation samples. The criteria for selection of the 48 genes were based on the genes from the 96-gene TLDA being highly correlated to their respective microarray data (r>0.70 comparing TLDA and microarray data) or if categorization of patients based on median level of expression of the TLDA gene demonstrated statistically significant prediction of PFS (univariate analysis P<0.01).

Heatmap was generated by obtaining fold-change values of each tumor over the average expression of all inflammation-related genes (Table 2) in children diagnosed <18 months with metastatic NBLNA. The data were winsorized (at 10% ile and 90% ile) to generate the heatmap.

In building the multivariate model, the association between each individual gene and probability of failure (i.e., having disease progression or not) was first examined by univariate logistic regression analysis, adjusting for age at diagnosis (Table 2). Genes with a P value of ≦0.25 were included in a multivariate logistic model, together with age at diagnosis as a variable. Backward selection was then used to exclude genes with a P value>0.10. All P values were obtained using likelihood ratio tests. For LOOCV analysis, each of the 133 CCG samples was iteratively excluded from the analysis cohort, and a prediction model was built with the remaining 132 samples as described above. The tumor-progression score for the excluded sample was then computed using the newly-built prediction model.

### Example 10

### NBL-NA Signature Accuracy

The relative contribution to the accuracy of the NBL-NA signature of each of the feature sets of the signature, i.e., age at diagnosis, the nine tumor cell-related genes, and five inflammation-related genes were assessed by comparing average AUC derived from 5000 permuted datasets.

To evaluate the contribution of inflammation-related genes to the accuracy of prediction, the inventors permuted the expression values of the five inflammation-related genes as a vector, without permuting age values or tumor-related gene expression values. This kept the correlation structure of these inflammation-related genes intact. The data for tumor cell-related genes and age at diagnosis were not changed. For each permutation, the inventors applied the original regression coefficients from the final prediction model to the permutated data, calculated the predicted logit scores, and then calculated the AUC for the permuted dataset. To evaluate the contribution of tumor cell-related genes to the accuracy of prediction, the inventors permutated the nine tumor genes as a vector without changing age and inflammation-related genes coefficients. The inventors then calculated the average AUC using the 5000 permuted datasets. To evaluate the contribution of tumor cell-related genes, the inventors permuted the nine tumor gene expression values as a vector without permuting age values or inflammation-related gene expression values, and again calculated average AUC using the 5000 permuted datasets. The inventors performed the analogous analysis by permuting age at diagnosis without permuting gene expression values to evaluate the contribution of age towards the prediction in our NBL-NA model.

The original accuracy of the prediction model for the 133 CCG samples as estimated by AUC was 0.9634. The average AUC of permuted age at diagnosis, permuted inflammation-related genes, and permuted tumor cell-related genes were 0.09070, 0.8418, and 0.6677, respectively. The inventors also permuted all the features (age at diagnosis, tumor cell related and inflammation related genes), which gave an average AUC of 0.4993 (˜0.50), as expected.

The overall AUC contribution from age at diagnosis, tumor cell-related, and inflammation-related genes together was 0.9634−0.5=0.4634. The relative contribution of a feature to the accuracy of the model was then computed as the percentage of the difference in the average permuted AUC for a given feature compared to the overall contribution of all features. Thus, age at diagnosis contributed (0.9634−0.9070)/0.4634×100%=12.2%, contribution of the inflammation genes was (0.9634−0.8418)/0.4634×100%=26.2%, and contribution of tumor-cell genes was (0.9634−0.6677)/0.4634×100%=63.8%. These values were rescaled to generate the final contributions so that on average 63%, 25%, and 12% of the accuracy was attributable to the tumor cell-related genes, inflammation-related genes, and age at diagnosis, respectively.

### Example 11

### Analysis for the Neuroblastoma Molecular Risk Group Classification Project

### A. Data

Sample 91207 was excluded from all analyses since all CT values were undetermined for this sample. After excluding sample 91207, TLDA data was available for a total of 133 CHLA samples and a total of 48 German samples. Gene IL17A was not detected in 97/133 CHLA samples and in 37/48 German Samples (Table 5). Therefore, IL17A was excluded from the analyses.

TABLE 5
Genes with undetermined CT's

| | | | Cumulative |
|---|---|---|---|
| Gene | # of Samples | Percent | Percent |
| CHLA | | | |
| CD19 | 5 | 4.07 | 4.07 |
| CX3CR1 | 12 | 9.76 | 13.82 |
| CXCL12 | 2 | 1.63 | 15.45 |
| IL17A | 97 | 78.86 | 94.31 |
| LOC284244 | 1 | 0.81 | 95.12 |
| MS4A1 | 2 | 1.63 | 96.75 |
| MYT1L | 1 | 0.81 | 97.56 |
| PTPN5 | 2 | 1.63 | 99.19 |
| SCN3A | 1 | 0.81 | 100 |
| German | | | |
| CAMTA1 | 1 | 2.56 | 2.56 |
| IGKC; IGKV1 | 1 | 2.56 | 5.13 |
| IL17A | 37 | 94.87 | 100 |

Note;
IL17A was excluded from all analyses given that CT of this gene was undetermined for most samples.

The distribution of the geometric mean of CT for the three housekeeping genes (GAPDH, HPRT1, SDHA) in CHLA or German samples was examined, and no extremely high geometric mean CT was found. After excluding gene IL17A, undetermined CT readings (with a value of 40) were very limited (Table 6), and there did not seem to be an association between undetermined CT readings and the geometric mean CT of housekeeping genes of the samples (Table 6).

TABLE 6
Samples that have Undetermined CT values (40)
(Gene IL17A excluded)

| | | | Geometric |
|---|---|---|---|
| | | | mean CT of 3 |
| Sample | Gene | CT | HK genes |
| CHLA | | | |
| 91257 | CD19 | 40 | 19.11 |
| 92174 | CX3CR1 | 40 | 20.76 |
| 86120 | CX3CR1 | 40 | 20.76 |
| 92218 | SCN3A | 40 | 21.58 |
| 92218 | MYT1L | 40 | 21.58 |
| 92218 | LOC284244 | 40 | 21.58 |
| 91017 | PTPN5 | 40 | 21.69 |
| 95003 | MS4A1 | 40 | 21.90 |
| 95003 | CD19 | 40 | 21.90 |
| 92085 | PTPN5 | 40 | 21.91 |
| 94049 | CD19 | 40 | 22.00 |
| 94049 | CX3CR1 | 40 | 22.00 |
| 94049 | MS4A1 | 40 | 22.00 |
| 96036 | CX3CR1 | 40 | 22.18 |
| 94050 | CX3CR1 | 40 | 22.18 |
| 86114 | CX3CR1 | 40 | 22.41 |
| 94189 | CX3CR1 | 40 | 22.88 |
| 94087 | CX3CR1 | 40 | 23.20 |
| 95259 | CX3CR1 | 40 | 23.23 |
| 94282 | CD19 | 40 | 23.36 |
| 94271 | CX3CR1 | 40 | 24.30 |
| 96015 | CX3CR1 | 40 | 25.46 |
| 96015 | CXCL12 | 40 | 25.46 |
| 94097 | CD19 | 40 | 26.75 |
| 94097 | CX3CR1 | 40 | 26.75 |
| 94097 | CXCL12 | 40 | 26.75 |
| German | | | |
| G12246 | CAMTA1 | 40 | 24.21 |
| G12246 | IGKC; IGKV1 | 40 | 24.21 |

The distribution of age at diagnosis (months) and the number of patients who experienced an event (progression/relapse/second malignancy/death from toxicity or treatment) in CHLA or German samples is given in Table 7.

TABLE 7
Sample Age (months) vs. Event

Event

| Age (in months) | No | Yes | Total |
|---|---|---|---|
| CHLA Samples | | | |
| 1-12 | 27 | 0 | 27 |
| 12-18 | 10 | 2 | 12 |
| >18 | 21 | 73 | 94 |
| Total | 58 | 75 | 133 |
| German Samples | | | |
| 1-12 | 0 | 4 | 4 |
| 12-18 | 5 | 0 | 5 |
| >18 | 13 | 25 | 39 |
| Total | 18 | 30 | 48 |

B. Determination of CT values

A CT value for each gene was determined as follows:

(1) Raw fluorescence values for each RT-PCR cycle were exported from the Applied Biosystems 7900HT Version 2.3 (Patch 1) Sequence Detection Systems software.
(2) For each gene within each sample, a baseline value was computed as the median of the fluorescence from cycles 3-15. To avoid overestimating the baseline for some high-expressing genes, the upper limit of this range was adjusted to a value that was a least 3 cycles lower than the computed CT value (below) for the gene. This adjustment was required only rarely, and resulted in only small adjustments to the originally computed CT values.
(3) The baseline value was subtracted from the raw fluorescence values, and a loess [1] smoothing function was fit. A CT value was computed as the point where the smoothed function intersected a fixed threshold value of 0.20. The assay was considered negative if the baseline-corrected function did not intersect the fixed threshold. In these cases the CT was assigned the value 40.

### C. Determination of the Gene Expression Score

Delta CT method was used to calculate the gene expression score. Though the delta CT method does not efficiently handle the issue of having censored data at the CT of 40, that should not significantly influence our analysis results since only a limited number of CT values were undetermined in our dataset (with a value of 40, Table 6).

D. Prediction model selection using the 133 CHLA tumors

Logistic regression models were used to determine the prediction model, with the outcome being the probability of having an event. Given that many genes on the TLDA cards were found to be correlated with age, we put age as a covariate in the prediction model so as to select genes that predict outcome independently from age. Age was treated as a continuous variable in the logistic regression analyses.

To build a prediction model, first the association between each individual gene with the outcome (having an event or not) was examined, adjusting for continuous age. Table 8 ranks the genes based on the significance of the association between each gene individually with event after adjusting for age. Genes with a p-value of <=0.25 were included in the multivariate logistic base model, together with continuous age. Then backward selection was used to exclude any genes with a p-value of >0.10. All p-values were from likelihood ratio tests. A total of 14 genes (PTPN5, GPATC4, H2AFV, FCGR3A; FCGR3B, CD14, PGM2L1, NTRK2, CD33, THAP2, IL6R, GFRA3, CAMTA1, IL10, BTBD3) together with age were included in the final prediction model (Table 9).

TABLE 8
Rank of Significance of the Association between Each of
the 44 Genes Individually with Event, after Adjusting for
Continuous Age (Logistic Regression)

| | | P Value from |
|---|---|---|
| Rank | Gene | Likelihood Ratio Test |
| 1 | H2AFV | 0.0001 |
| 2 | GPATC4 | 0.0003 |
| 3 | PTPN5 | 0.0006 |
| 4 | PGM2L1 | 0.0017 |
| 5 | GNAI1 | 0.0018 |
| 6 | TMEFF1 | 0.0030 |
| 7 | IGKC; IGKV1 | 0.0048 |
| 8 | FCGR3A; FCGR3B | 0.0058 |
| 9 | NXPH1 | 0.0212 |
| 10 | CNR1 | 0.0230 |
| 11 | C5orf13 | 0.0265 |
| 12 | TGFB1 | 0.0283 |
| 13 | GPR85 | 0.0360 |
| 14 | CD33 | 0.0392 |
| 15 | HOXC6 | 0.0487 |
| 16 | GFRA3 | 0.0501 |
| 17 | THAP2 | 0.0542 |
| 18 | NTRK1 | 0.0612 |
| 19 | SMARCE1 | 0.0617 |
| 20 | IL6 | 0.0631 |
| 21 | BTBD3 | 0.0634 |
| 22 | CXCL12 | 0.0672 |
| 23 | IL6R | 0.0830 |
| 24 | CAMTA1 | 0.1034 |
| 25 | C6orf168 | 0.1406 |
| 26 | CD14 | 0.1498 |
| 27 | YPEL1 | 0.2010 |
| 28 | ST7 | 0.2157 |
| 29 | IL10 | 0.2193 |
| 30 | NTRK2 | 0.2338 |
| 31 | MS4A1 | 0.2883 |
| 32 | MYT1L | 0.2918 |
| 33 | C1orf35 | 0.2941 |
| 34 | PARP6 | 0.3671 |
| 35 | STAT3 | 0.3716 |
| 36 | CD19 | 0.3819 |
| 37 | PRG2 | 0.3949 |
| 38 | HRK | 0.4326 |
| 39 | PAK7 | 0.4394 |
| 40 | CX3CR1 | 0.7708 |
| 41 | APBA2 | 0.8091 |
| 42 | LOC284244 | 0.8500 |
| 43 | SOX4 | 0.9115 |
| 44 | SCN3A | 0.9456 |

TABLE 9
The Final Prediction Model (CHLA Patients)

| | | 95% CI | | 95% CI | Likelihood | Coefficient | |
|---|---|---|---|---|---|---|---|
| | | of the | Odds | of the | ratio test | adj. only | LRT |
| Variable | Coefficient | Coefficient | Ratio | Odds Ratio | p value | age | p value |
| Intercept | −3.38 | (−12.25, 5.48) | | | | | |
| Age at | 0.62 | (0.18, 1.06) | 1.86 | (1.20, 2.89) | 0.0006 | | |
| Diagnosis | | | | | | | |
| (in years) | | | | | | | |
| PTPN5 | 0.72 | (0.35, 1.08) | 2.05 | (1.42, 2.95) | <0.0001 | 0.24 | 0.0006 |
| GPATC4 | 1.79 | (0.74, 2.84) | 5.99 | (2.09, 17.18) | <0.0001 | 0.73 | 0.0003 |
| H2AFV | −2.06 | (−3.29, −0.82) | 0.13 | (0.04, 0.44) | <0.0001 | −0.86 | 0.0001 |
| FCGR3A; | 1.56 | (0.62, 2.49) | 4.75 | (1.87, 12.08) | <0.0001 | 0.31 | 0.0058 |
| FCGR3B | | | | | | | |
| CD14 | −2.52 | (−4.06, −0.99) | 0.08 | (0.02, 0.37) | 0.0001 | 0.22 | 0.1498 |
| PGM2L1 | −1.36 | (−2.46, −0.26) | 0.26 | (0.09, 0.77) | 0.0053 | −0.49 | 0.0017 |
| NTRK2 | −0.65 | (−1.19, −0.11) | 0.52 | (0.30, 0.90) | 0.0119 | 0.15 | 0.2338 |
| CD33 | 1.28 | (0.13, 2.43) | 3.60 | (1.14, 11.38) | 0.0137 | 0.30 | 0.0392 |
| THAP2 | −1.60 | (−3.11, −0.10) | 0.20 | (0.05, 0.90) | 0.0207 | −0.53 | 0.0542 |
| IL6R | 1.18 | (−0.01, 2.36) | 3.24 | (0.99, 10.59) | 0.0327 | 0.23 | 0.0830 |
| GFRA3 | −0.43 | (−0.86, 0.004) | 0.65 | (0.42, 1.00) | 0.0437 | −0.24 | 0.0501 |
| CAMTA1 | 0.71 | (−0.02, 1.44) | 2.03 | (0.98, 4.21) | 0.0499 | −0.23 | 0.1034 |
| IL10 | −0.91 | (−1.95, 0.13) | 0.40 | (0.14, 1.14) | 0.0601 | 0.15 | 0.2193 |
| BTBD3 | 0.85 | (−0.13, 1.84) | 2.35 | (0.88, 6.29) | 0.0708 | −0.28 | 0.0634 |

Note:
Coefficients/Odds ratios for genes are with respect to a 2 fold increase in gene expression (i.e., a change of 1 delta CT).

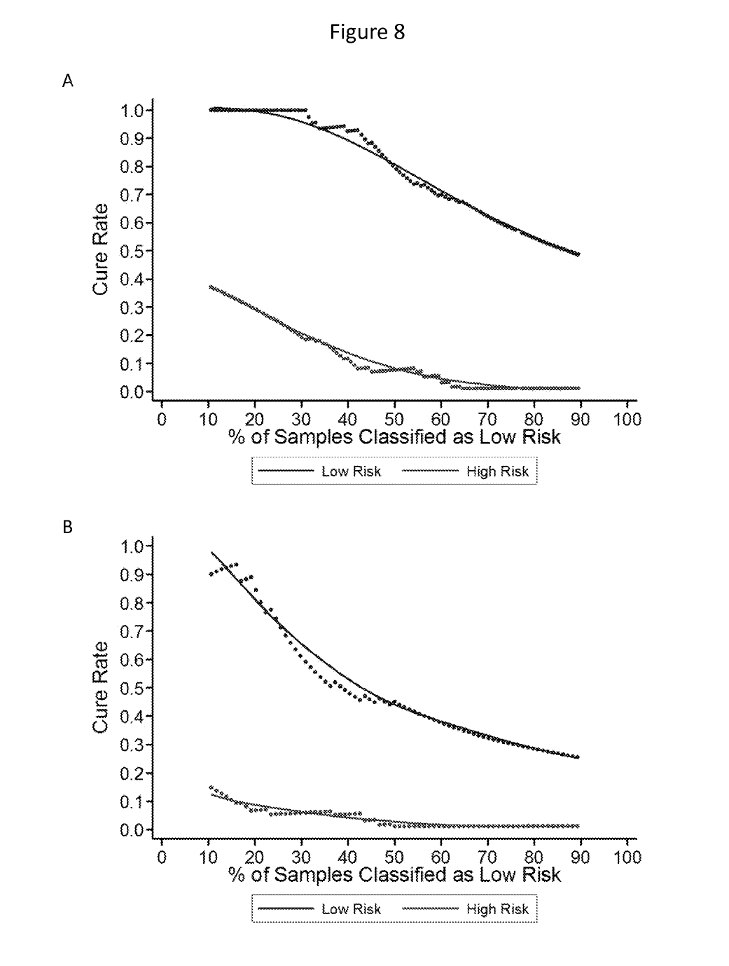

Prediction results using the final prediction model for the CHLA Samples are shown in FIGS. 6, 7 and 8.

### F. Leave One Out Cross Validation (LOOCV) of the Prediction Model: CHLA Samples

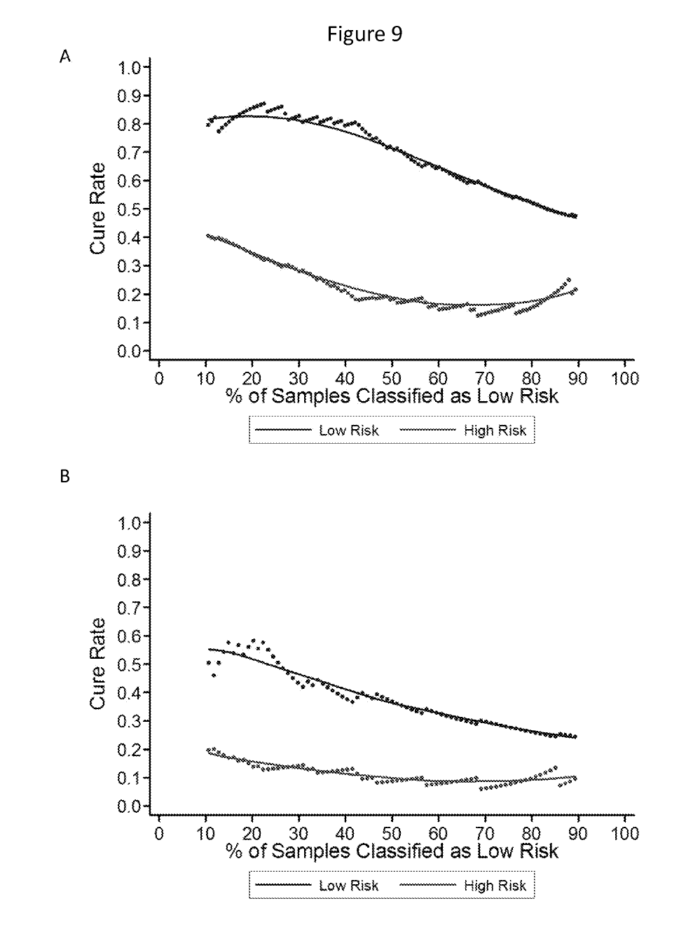

Internal validation of the prediction model was done with the LOOCV approach. Each of the 133 CHLA samples was iteratively excluded from the analysis cohort, and a prediction model was built with the remaining 122 samples using the same procedure described in the prediction model selection section, and the probability of failure (having an event) for the sample that was left out was predicted using the newly built prediction model. The results of the LOOCV can be found in FIGS. 9A and 9B.

### G. External Validation of the Prediction Model Using the 48 German Samples

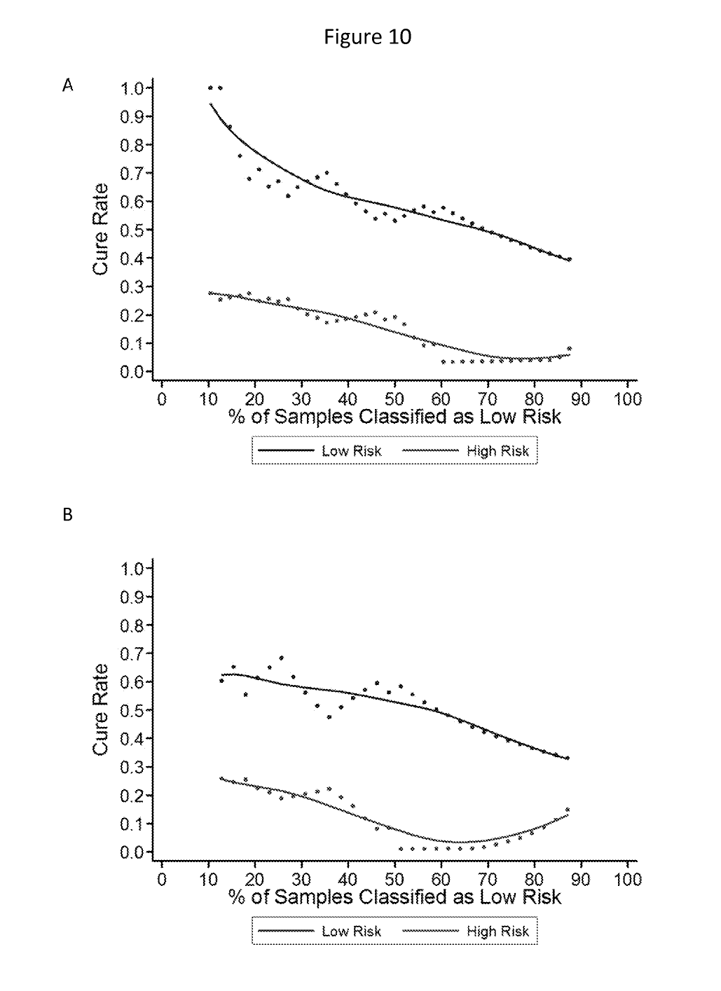

The fourteen genes+continuous age prediction model was applied to the German samples, and the prediction results can be found in FIGS. 2 and 10.

### Example 12

44 paired bone marrow (BM) and blood samples were obtained from patients on COG ANBL0532/ANBLOOB1 (diagnosis n=8), COG ANBL0032/ANBL0931 (pre-study n=9, post-study n=25, relapse n=2). Higher DG indicates lower tumor content, with each unit increase equaling ˜0.3 log lower tumor content. A DG score of 40 indicated a negative result. The overall frequency of positivity in BM was 33 (75%), while positivity in blood was 17 (38.6%). BM DG was correlated with blood DG (rank correlation 0.46, p=0.002). Blood signals were less than BM signals (4.7 Ct difference in DGs), but the difference ranged from 8.3 in patients with the BM DG<30 to 1.8 in those with BM DG>35. Only 3 patients (6.8%) whose blood was positive (mean DG 39.4) had undetectable tumor in BM, while in 19 patients (43.1%) BM was positive (mean DG 38.8) but blood was negative.

### Example 13

Expression of CHGA, DCX, DDC, PHOX2B, and TH (neuroblastoma genes) and of B2M, GAPDH, HPRT1, and SDHA (housekeeping genes) was quantified with TLDA. Results were reported as positive/negative for tumor and as the geometric mean Cycle Threshold for the five genes (DG=detection gene score, which is inversely related to tumor content). TLDA was performed on 16 BM and 7 blood samples from 17 patients with recurrent/refractory neuroblastoma. The number of 123I-MIBG avid sites, the longest tumor dimension (LD) by CT/MRI, and BM tumor cells by morphology (positive/negative, percentage) were scored by central review of radiology and pathology reports.

The TLDA assay detected tumor cells in 14/16 (87.5%) BM and 4/7 (57%) blood specimens. For 6 BM/blood samples obtained <6 days apart, the average DG was 30.9 in BM and 37.3 in blood. For all patients, TLDA detected tumor in 11/12 MIBG+ and 3/4 MIBG−, 8/9 CT/MRI+ and 5/7 CT/MRI−, and 9/9 BM+ and 5/7 BM− by morphology. TLDA and morphologic detection of tumor cells in BM (n=16) were correlated when BM was positive (p=0.028) and with percent BM tumor cells (p=0.0074). There was no correlation with numbers of MIBG sites or of tumor LD.

### Example 14

Expression of CHGA, DCX, DDC, PHOX2B, and TH (neuroblastoma genes) and of B2M, GAPDH, HPRT1, and SDHA (housekeeping genes) was quantified with a TLDA assay. Results are reported as positive/negative for tumor cells and as the geometric mean Cycle Threshold expression of the five genes (DG=detection gene score). Patients in studies CCG-321P3, CCG-3891, and 91LA6 had not developed progressive disease at 3 and 9 months after myeloablative therapy when BMs were obtained.

Neuroblastoma genes are strongly expressed by untreated neuroblastoma tumors (n=25) and multi-drug sensitive/resistant neuroblastoma cell lines (n=23) but not detectibly or weakly by BM (n=20), peripheral blood mononuclear cells (n=25), or peripheral blood stem cells (n=15) from normal adults. Sensitivity is 81% for detecting one tumor cell per 106 normal cells. TLDA and immunocytology results were highly correlated when immunocytology was positive (r=−0.93, p<0.001), but TLDA identified tumor cells when immunocytology was negative. Tumor cells were detectible in 87% and 76% of BMs at 3 (n=46) and 9 (n=38) months after myeloablative therapy, and subsequent EFS correlated with tumor load (P=0.02 at 3 and <0.001 at 9 months). At these same times, DG<37 vs. DG=40 predicted 1.8- and 3.7-fold increases in disease progression.

### Example 15

### Preparation of High Quality RNA from Frozen and Fresh Specimens

Total RNA was prepared from ≧2×10⁷ fresh or viably frozen mononuclear cells using QIAGEN RNeasy® Mini Kit for fresh samples, but TRIzol Reagent and further processed with the QIAGEN RNeasy® Mini Kit for frozen samples. RNA is analyzed with the Agilent Bioanalyzer to obtain an RNA Integrity Number (RIN), and specimens with RIN values of ≧5 initially were used for the TLDA assay. Two-step RT-PCR was performed using Oligo-dT+gene specific primers (CHGA, DCX, DDC, and TH) for cDNA synthesis. Five detection, 39 microenvironment, and four housekeeping genes were analyzed using inventoried TaqMan® Gene Expression Assays that were pre-designed and pre-optimized probe and primer sets for quantitative gene expression analysis (Table 10). Cards with these genes were manufactured by Applied Biosystems under standard conditions and shipped to the inventors' laboratory. A standard RNA preparation consisting of PBMC RNA with a final dilution of SK-N-BE2 neuroblastoma cell line RNA of 10⁻⁴ were included as a batch control each time new cards were received. Universal cycling conditions were used to enable assays for all genes to be run together with a single card using the 7900HT instrument. Sensitivity was optimal when 2,500 ng of cDNA was loaded into each port of the TLDA microfluidics card. Specimen and lab variability were controlled by RIN and housekeeping genes.

### Example 16

### Highly Sensitive Quantification of “Tumor Load” from Expression of Five Genes that are Tumor Specific in Bone Marrow and Blood

The sensitivity for detecting neuroblastoma cell line RNA spiked into PBMC RNA was determined because of inherent inaccuracies with seeding few neuroblastoma cells into PBMC. 44 assays were performed using RNA from 8 cell lines at ratios of 10⁻⁴ to 10⁻⁷. The probability of detecting 10⁻⁶ was 0.5 and 10⁻⁵ was 0.9.

Estimation of neuroblastoma cell detection sensitivity in a sample predicts 85% sensitivity when RNA is prepared from 20 million cells and when 1 neuroblastoma cell is present among 10⁶ normal cells.

Detection sensitivity with five genes (including TH) is superior to that of a single gene (TH). The five-gene detector has nearly 100% sensitivity to detect neuroblastoma RNA at a dose of 10⁻⁵, whereas the TH-only detector has sensitivity of under 60%. In terms of NB cell detection, the 5-gene signature can detect a 10⁻⁶ neuroblastoma cell frequency in PBMC with 81% probability compared to under 30% for a TH-alone detector.

RNA quality as defined by housekeeping gene expression has a notable effect on detection sensitivity, especially at neuroblastoma cell frequencies of 10⁻⁶ or lower, when the housekeeping gene signal increases by 1 and 2 cycles (Ct). However, sensitivity of the assay remains very good at neuroblastoma cell levels of 10⁻⁵ and higher.

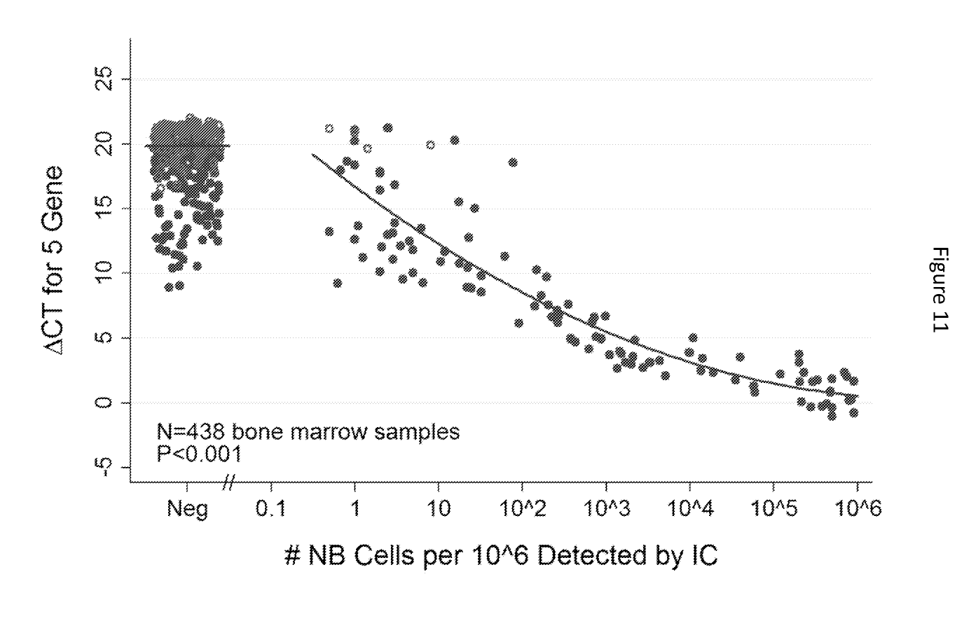

In comparing sensitivity of the TLDA assay vs. immunocytology for detecting tumor cells in patient BM and PBSC samples, the TLDA assay classified a significantly higher number of samples as positive (FIG. 11). When immunocytology is positive, the two assays are highly correlated.

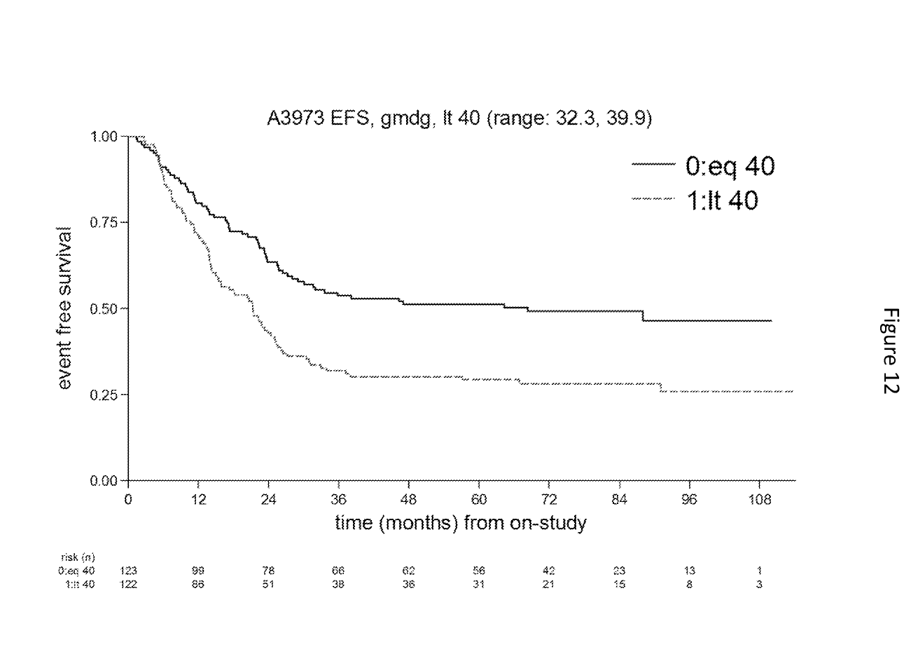

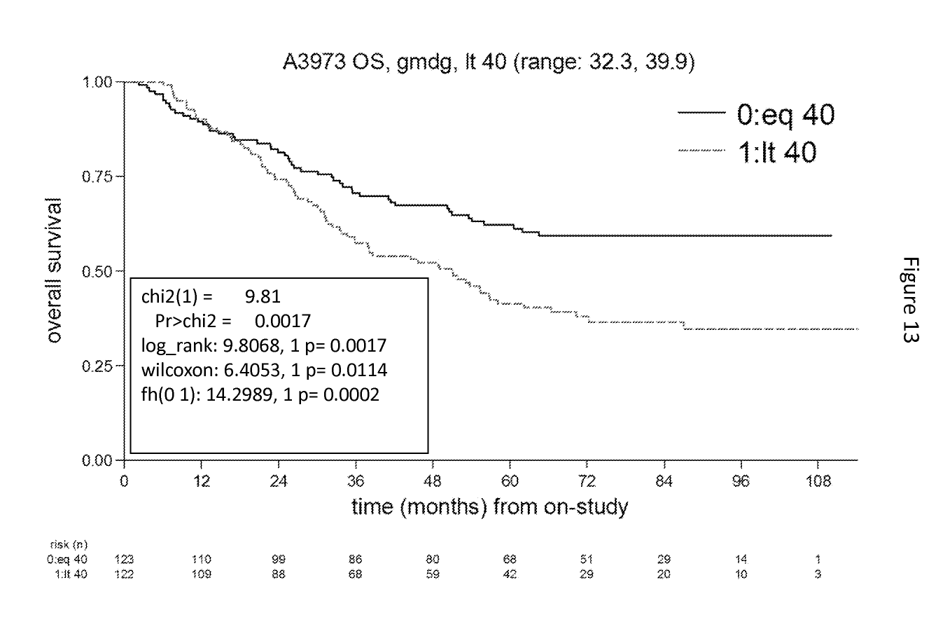

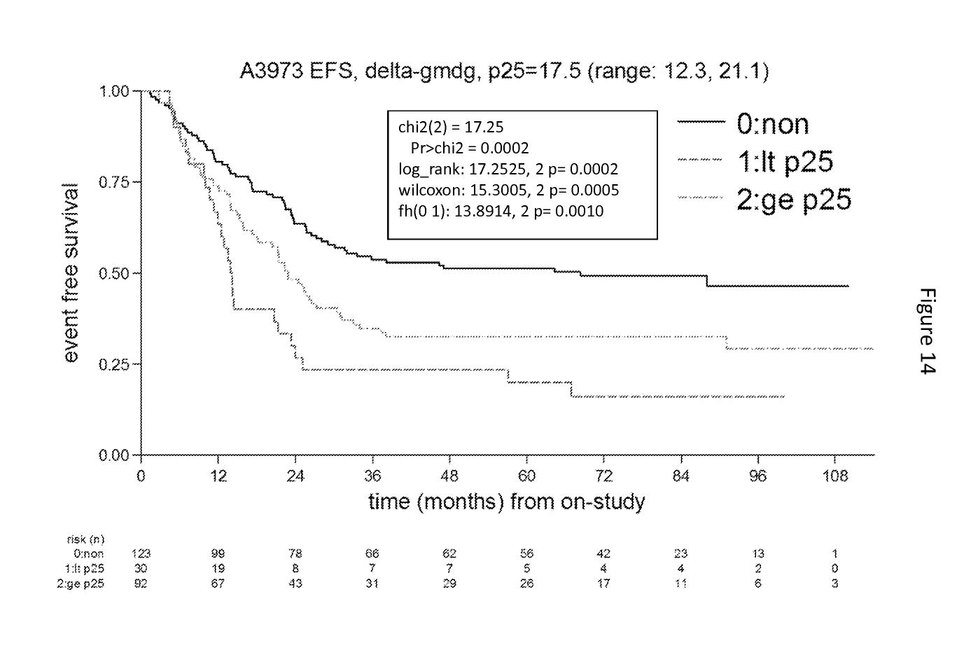

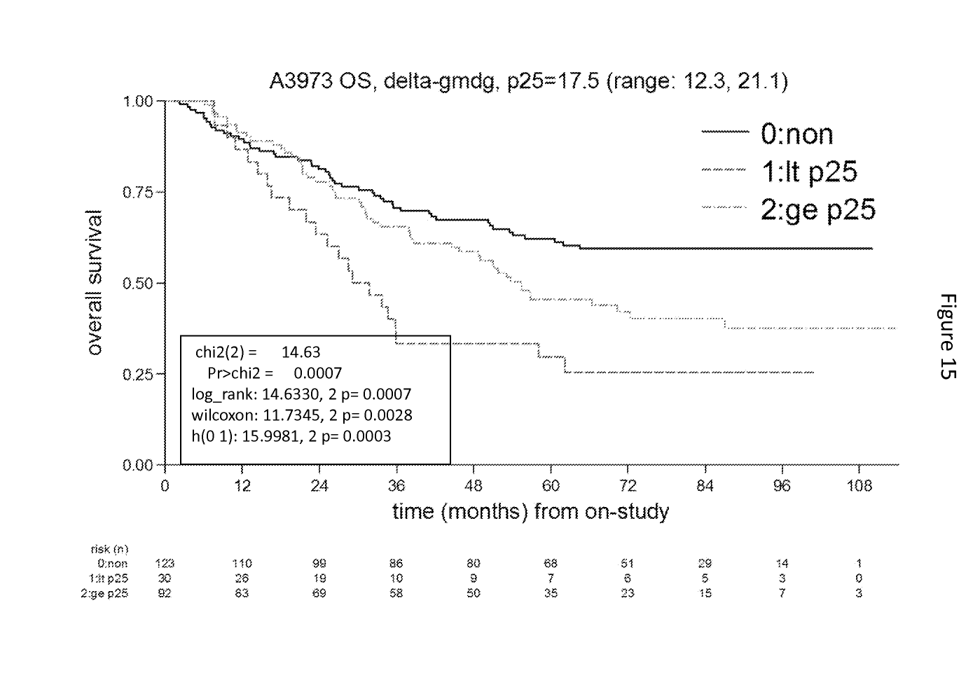

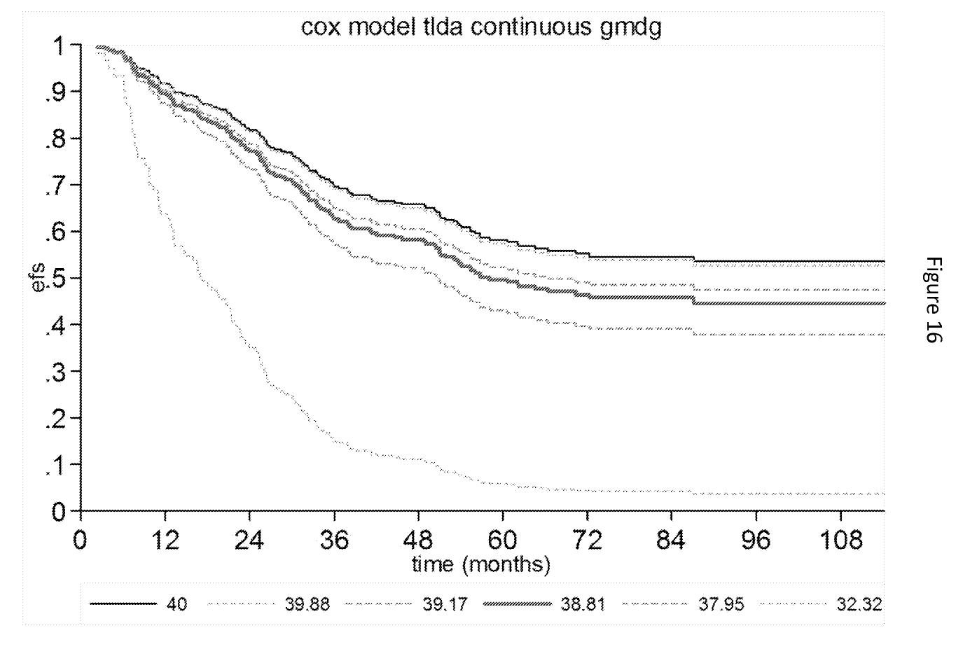

The inventors assessed PBSC with a 5-gene TLDA assay to determine frequency of positive tests; correlation of positivity with EFS/OS; and effect of purging. 463 of 486 patients had PBSC collected, and 372 were transplanted. Immunocytology detected tumor cells in 4/460 day 1 PBSC and in none after purging. Among 245 patients with day 1 samples available for TLDA analysis, 122 (50%) were positive: 68/129 (53%) from purged and 54/116 (47%) from non-purged arms. Five-year EFS correlated with the DGS: 20%±7% for DGS<25^{th} percentile; 33%±5% for DGS≧25^{th} percentile; 51%±5% for non-detectible (P=0.0002) (positive DGS range=32.3-39.9). Five-year OS paralleled EFS (30%±8%, 45%±5%, and 62%±4%, respectively; P=0.0007). The 245 patients whose PBSC were evaluated with TLDA were representative of all 486 patients. TLDA analysis of 52 before/after purging pairs of PBSC showed that pre-purge positive signals became negative (52%), weaker (28%), or slightly stronger (20%). Five-gene TLDA analysis of PBSC provides a prognostic biomarker that likely reflects the early response to induction chemotherapy (FIGS. 12-16).

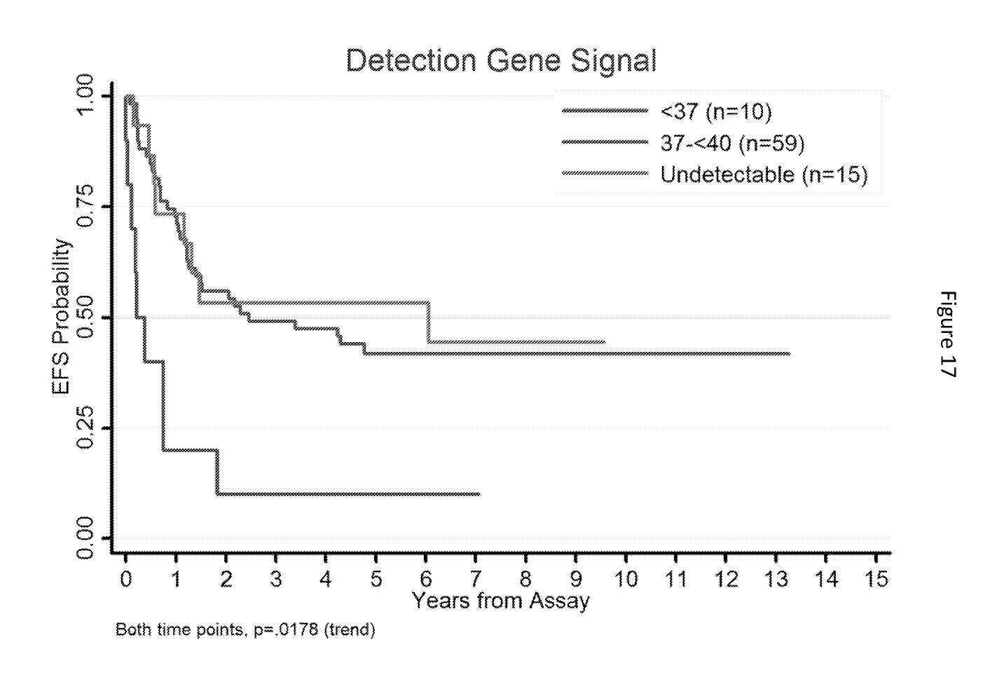

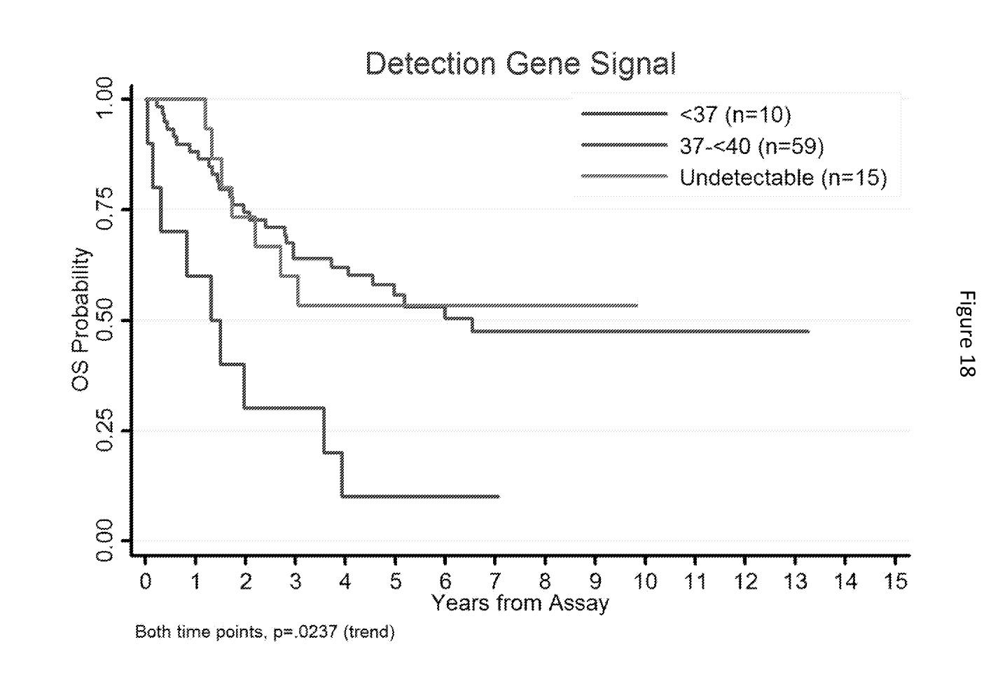

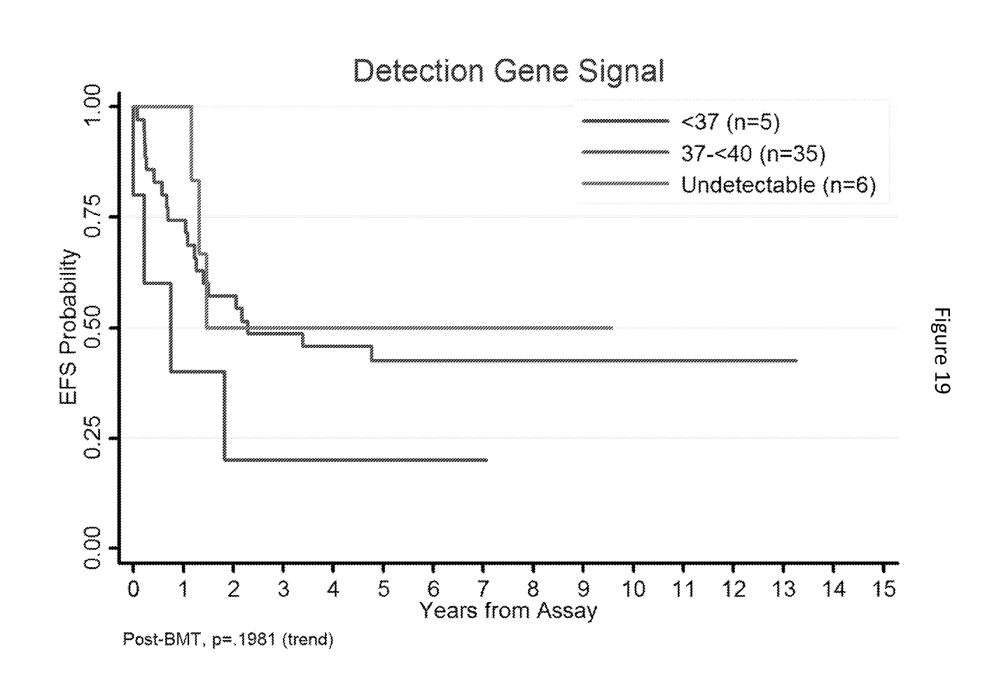

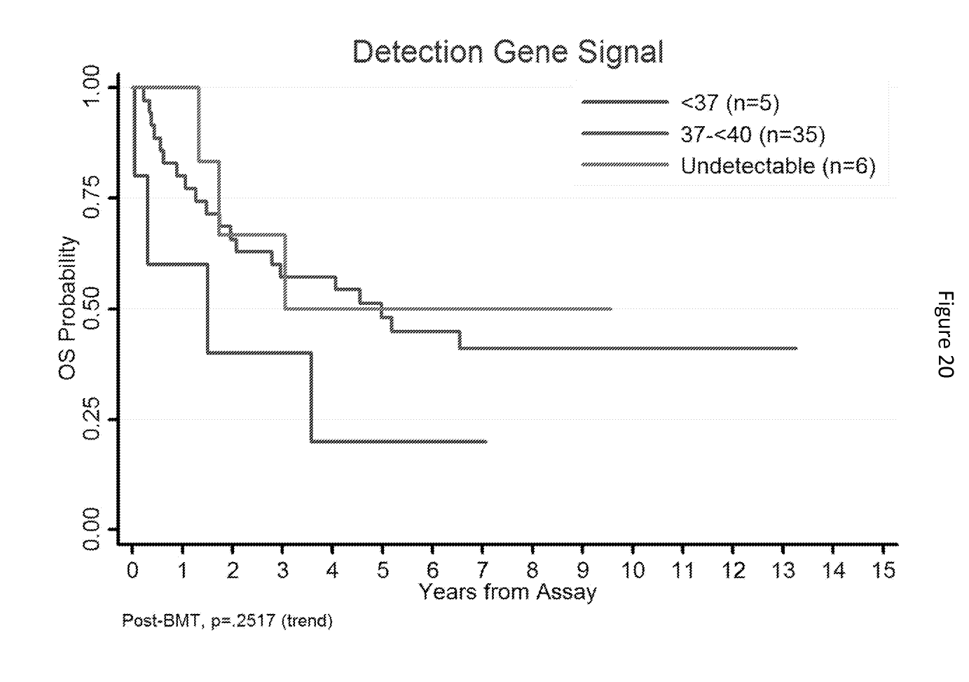

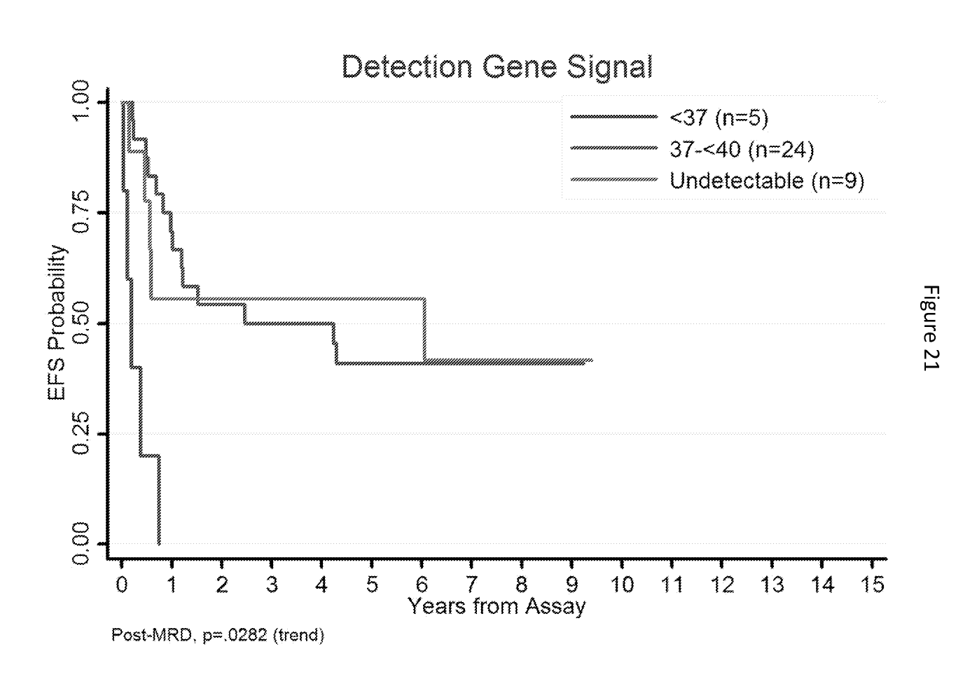

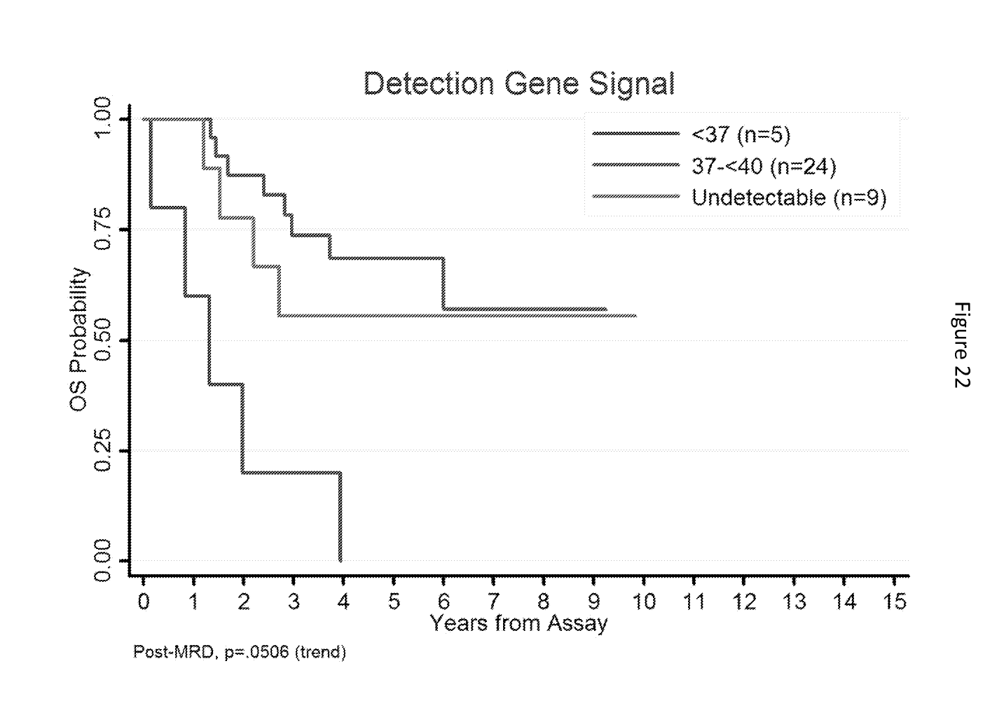

The five-gene TLDA assay also identified patients at high-risk for disease progression after myeloablative consolidation therapy. Tumor cells were detectible in 87% and 76% of BMs at 3 (n=46) and 9 (n=38) months after myeloablative therapy, and subsequent EFS correlated with tumor load (P=0.02 at 3 and <0.001 at 9 months). At these same times, DG<37 vs. DG=40 predicted 1.8- and 3.7-fold increases in disease progression. This 5-gene TLDA assay provides a sensitive and quantitative test for neuroblastoma cells in bone marrow that identifies patients at high-risk for disease progression (FIGS. 17-22).

TLDA assay also detected neuroblastoma cells in both BM and blood in patients with recurrent/refractory neuroblastoma at high rates, and that it frequently detects tumor cells when BM morphology and imaging evaluations do not.

### Example 17

### Quantification of Expression of 39 Genes in Bone Marrow and Blood by Normal Cells that May Affect Tumor Cell Growth and Survival and that May be Therapeutic Targets

The 39 genes were chosen because, in general, they reflect immunology, inflammation, and angiogenesis pathways/functions that may influence tumor cell proliferation, survival, and dissemination. These genes were included on the TLDA card with the neuroblastoma and housekeeping genes so that one assay simultaneously evaluates expression of 48 genes. RNA from mononuclear cells from marrow, blood, and PBSC of normal adult donors and from neuroblastoma cell lines was evaluated with the TLDA assay. Expression of the 39 genes primarily reflects normal cells, although some genes (TGFB1, VEGFA, CXCR4, and GATA3) are expressed at very high levels by both normal and neuroblastoma cells. These genes distinguish bone marrow obtained 3 months after myeloablative therapy and hematopoietic stem cell transplant from marrow obtained 9 months afterward, indicating that the marrow microenvironment changes with time after transplant.

### Example 18

Expression by 39 genes by normal cells in bone marrow and blood is different, and so the assay provides molecular information about the quality of the bone marrow sample (i.e., whether it is contaminated by excessive blood cells, which can impact the ability to identify tumor cells). Analyses of blood vs. bone marrow demonstrate that the 39 microenvironment gene signature can distinguish the two types of specimens. Key genes for this distinction include CD4, CD8, and CD34.

### Example 19

### Method for Calculating Test Results has been Improved Compared to What is Commercially Available

A CT value for each gene was determined as follows: Raw fluorescence values for each RT-PCR cycle were exported from the Applied Biosystems 7900HT Version 2.3 (Patch 1) Sequence Detection Systems software. For each gene within each sample, a baseline value was computed as the median of the fluorescence from cycles 3-15. To avoid overestimating the baseline for some high-expressing genes, the upper limit of this range was adjusted to a value that was a least 3 cycles lower than the computed CT value (below) for the gene. This adjustment was required only rarely, and resulted in only small adjustments to the originally computed CT values. The baseline value was subtracted from the raw fluorescence values, and a linear spline function was fit. A CT value was computed as the point where the spline function intersected a fixed threshold value of 0.5. The assay was considered negative if the baseline-corrected function did not intersect the fixed threshold. In these cases the CT was assigned the value 40.

The NB detection score is the geometric mean of CT values of the set of 5 detection genes. If all detection genes were negative (i.e., CT=40), the assay was considered negative for neuroblastoma. The geometric mean of the four housekeeping genes serve as a measure of RNA quality as well as assay validity and is used to adjust the NB detection score by the ΔCT method or other methods, depending on the context.

TABLE 10
Genes for which expression is quantified using the TLDA assay

| | | | | | | | |
|---|---|---|---|---|---|---|---|
| Housekeeping | B2M | GAPDH | HPRT1 | SDHA | | | |
| Detection | CHGA | DCX | DDC | PHOX2B | TH | | |
| Micro- | CD14 | CD16 | CD163 | CD19 | CD34 | CD4 | CD40LG |
| environment | | (FCGR3B; | | | | | |
| | | 3A) | | | | | |
| | CD86 | CD8A | CSF1 | CSF1R | CTLA4 | CX3CR1 | CXCL12 |
| | | | (M-CSF) | (CD115) | | | |
| | CXCR3 | CXCR4 | FLT1 | FOXP3 | GNLY | GZMB | HMOX1 |
| | | | (VEGFR1) | | | | |
| | IFNG | IL10 | IL13 | IL15 | IL2RA | IL4 | IL6 |
| | IL6R | IL7 | IL7R | IL8 | KDR | KLRK1 | NCAM1 |
| | | | | | (VEGFR2) | (NKG2D) | |
| | TBX21 | TEK | TGFB1 | | VEGFA | | |

### Example 20

### Processing of cDNA on TLDA Assay

The TaqMan Low Density Array (TLDA) is a 384-well micro fluidic card that enables performance of 384 simultaneous real-time PCR reactions without the need to use liquid-handling robots or multichannel pipettes to fill the card. This low- to medium-throughput micro fluidic card allows for 1-8 samples to be run in parallel against 12-384 TaqMan® Gene Expression Assay targets that are pre-loaded into each of the wells on the card. The TaqMan® Low Density Array is completely customizable. Over 47,000 TaqMan® Gene Expression Assays that are designed for human, mouse, and rat genes are available for incorporation into cards.

Specimen—cDNA prepared from RNA of blood or bone marrow cells, 50 ng/μl

### Reagent—TaqMan 2× Universal PCR Master Mix (No AmpErase UNG; 4324018).

Equipment and Supplies—TaqMan Low Density Array (TLDA) Microfluidics Cards, Microfluidics Card specific thermal block, Microfluidics Card specific custom buckets and adaptors for Sorvall Legend RT centrifuge (75015679), Microfluidics Card Sealer (Model 4331770), Vortex, Microcentrifuge (Eppendorf, model5430R), Scissors.
Procedure—Bring the Microfluidics Card from refrigerator (2˜8° C.) to room temperature, and let sit for 15-30 minutes. Install Microfluidics Card specific thermal block into 7900HT. Remove labeled 0.5 ml microfuge tubes with cDNA samples from the −20° C. freezer and thaw at room temperature. Add the following components to the labeled 0.5 ml tube:

| Components | Volume per reaction (μl) |
|---|---|
| cDNA sample + RNase/DNase-free water | 50 |
| TaqMan 2X Universal PCR Master Mix | 50 |
| (No AmpErase UNG) | |
| Total Volume | 100 |

Cap the tubes and thoroughly mix the solution by gentle vortexing2times. Centrifuge the microcentrifuge tubes to eliminate air bubbles from the mixture (12,000 rpm, 10 sec). Load 100 μl of the sample into a port of the Microfluidics Card slowly (8 ports per card). Centrifuge the Microfluidics Card at 1200 rpm, for 1 min at 20° C. by the Card specific centrifuge. Repeat two times. Seal the Card and cut the loading part of the Card with scissors. Only go up once; do not go back. Aluminum side up. Start SDS 2.2.2 software>File>New>384 TLDA ΔΔCT>Microdectionbatch #>type in Barcode. Select rows for the wells and type in sample ID. Place the Microfluidics Card into the Applied Biosystems 7900HT system and run the Card.

### Example 21

### RNA Protection and Extraction for Frozen Samples

The purpose is for the protection and isolation RNA from frozen bone marrow, blood, and PBSC samples. TRIzol reagent is a ready to use mixture of phenol, guanidine isothiocyanate, red dye and other proprietary components that can be used to isolate total RNA in a single step. DNA and proteins can be removed with sequential precipitation from the organic phase. The red dye allows easy detection of the organic phase and is non-interactive with nucleic acids.

Immediately before TRIzol lysis, samples are defrosted in RNAprotect Cell Reagent which can protect RNA in cells instantly during thawing process. This innovative defrosting method greatly improves the RNA quality comparing to the traditional defrosting method.

### Specimen—Frozen PBSC, Blood, and Bone Marrow

Reagents—RNAprotect Cell Reagent (QIAGEN, Cat. No. 76526; size 250 ml); TRIzol Reagent (Invitrogen, Cat. No. 15596-018; size 200 ml); Chloroform (Sigma-Aldrich, Cat. No. C-2432-500 ml); Isopropyl alcohol (Sigma-Aldrich, Cat. No. I-9516-500 ml); 100% and 75% Ethanol (in RNase-free water); RNase-free water, buffer RLT from QIAGEN, RNeasy® Mini Kit (250) (Cat #74106); β-Mercaptoethanol (Sigma, M-6250)
Equipment and Supplies—Pipettes for use with only RNA; Microcentrifuge (Eppendorf, model 5430R, Fisher Scientific, Cat. No. 05413804) Eppendorf 6×15/50 ml conical rotor w/lid (5430) (Fisher Scientific, Cat. No. 05401512); 37° C. water bath; QIAGEN, RNeasy® Mini Kit (250) (Cat #74106); 15 ml polypropylene, sterile tubes; Nuclease free microcentrifuge tubes and tips.
Procedure—Aliquot 2.5 volumes of RNAprotection Cell Reagent at room temperature to a 15 ml polypropylene tube for each sample. Carefully unscrew and take the cap off the frozen sample vial (if cap is stuck with ice, warm the cap with fingers till it opens with ease). Place frozen sample vial in 37° C. water bath for 20 seconds to let the liquid nitrogen evaporate. Defrost the sample with 2.5 volumes of RNAprotect Cell Reagent by pipetting the reagent directly to the frozen samples vial and transferring back to the 15 ml tube, repeat several times till completely thawed. Centrifuge at 2,500×g for 5 minutes in a microcentrifuge (e.g., eppendorf, model 5430R) at room temperature with a special 15/50 ml conical rotor. Or transfer the cell suspension into several 1.5-2.0 ml microtubes and spin in a regular microcentrifuge tube rotor. Remove supernatant completely without disturbing the pelleted cells. Add total 1 ml of TRIzol Reagent to the pelleted cells. Lyse the cells by pipetting, and transfer the lysate to a new 1.5-2.0 ml microtube. Incubate 5 minutes at room temperature. Add 300 μL of Chloroform. Shake vigorously for 1 min, then let stand for 3 min at room temperature. Centrifuge at 14,000 rpm for 15 min at 4° C. Transfer the upper aqueous phase to new 1.5 ml microfuge tube. Add 500 μL of Isopropyl alcohol. Mix well, and then let stand for 10 min at room temperature. Centrifuge at 14,000 rpm for 20 minutes at 4° C. Remove the supernatant by pouring off solution into a waste collection tube; quick spin and completely remove liquid with a P200 pipette. Add 1 ml of 75% ice-cold ethanol, and centrifuge at 14,000 rpm for 2 minutes at 4° C. Repeat one more time. Remove the supernatant by pouring off solution into a waste collection tube; quick spin and completely remove liquid with a P200 pipette. Air-dry the pellet (approx. 3 minutes). Do not over dry. Add 100 μL of RNase-free water, vortex and spin (can stop here, and store at −80° C. freezer). Add 350 μL RLT buffer with 1% β-Mercaptoethanol, mix and spin. Add 250 μL 100% ethanol, pipet and transfer immediately to an RNeasy spin column. Continue with Proc-9 Isolation of RNA Using RNeasy mini kit protocol.

### Example 22

Purpose is for the isolation total RNA from fresh PBSC, blood and bone marrow. The RNeasy® Mini Kit is used to purify total RNA from animal cells, animal tissues, and yeast, and for cleanup of RNA from crude RNA preps and enzymatic reactions (e.g., DNase digestion). The RNeasy Kits are designed to purify RNA from small amounts of starting material. They provide a fast and simple method for preparing up to 100 μg total RNA per sample. The purified RNA is ready for use in downstream applications such as real-time (TaqMan) RT-PCR. The RNeasy procedure represents a well-established technology for RNA purification. This technology combines the selective binding properties of a silica-based membrane with the speed of microspin technology. A specialized high-salt buffer system allows up to 100 μg of RNA longer than 200 bases to bind to the RNeasy silica membrane. Biological samples were first lysed and homogenized in the presence of a highly denaturing guanidine-thiocyanate—containing buffer, which immediately inactivates RNases to ensure purification of intact RNA. Ethanol was added to provide appropriate binding conditions, and the sample was then applied to an RNeasy Mini spin column, where the total RNA binds to the membrane and contaminants were efficiently washed away. High-quality RNA was then eluted in 12-45 μl water.

### Specimen—Fresh PBSC, Blood and Bone Marrow.

### Reagents—QIAGEN, RNeasy® Mini Kit (250) (Cat #74106); QIAGEN, RNase-Free DNase Set (50) (Cat #79254); QIAGEN, QIAshredder (250) (Cat #79656); β-Mercaptoethanol (Sigma, M-6250); 70% Ethanol (ETOH).

Equipment and Supplies—Pipettes and tips that are only used for RNA; Nuclease-free 1.5 ml microcentrifuge tubes; Microcentrifuge (Eppendorf model 5430R, Fisher Scientific, Cat. No. 05413804).
Procedure—Label the QIAshredder columns (purple) and the RNeasy columns (pink) with specimen ID. Transfer the mixture (viscous) to QIAshredder for homogenization. Centrifuge in Eppendorf microcentrifuge at 14,000 rpm for 2 min. Add 600 μl of 70% EtOH to the flow-through. Mix by pipetting. Load up to 700 μl of the sample to an RNeasy spin column. Centrifuge at 10,000 rpm for 20 sec. Discard the flow-through. Load remaining sample up to 700 μl successively onto the RNeasy column, and centrifuge as above. Add 450 μl Buffer RW1 to wash the spin column. Centrifuge at 10,000 rpm for 20 sec to wash the column. Discard the flow-through. Add 80 μl DNase I solution (10 μl DNase I stock+70 μl Buffer RDD) directly onto the silica-gel membrane of the spin column. Incubate for 15 min at room temperature. Add 450 μl Buffer RW1. Centrifuge at 10,000 rpm for 20 sec. Transfer the column into a new 2 ml collection tube. Add 500 μl Buffer RPE to wash the column. Centrifuge at 10,000 rpm for 20 sec. Discard the flow-through. Add another 500 μl Buffer RPE. Centrifuge at 10,000 rpm for 2 min to dry the membrane. Cut off caps of 1.5 ml sterile tubes for the next centrifugation step. Transfer the RNeasy spin column to a 1.5 ml tube with its cap-removed. Centrifuge at 14,000 rpm for 1 min. Transfer the RNeasy spin column to QIAGEN 1.5 ml tube with cap. Remove remaining liquid from the ring of the column with a 10 μl pipette. Label tube with specimen ID and date. Add 12-45 μl of RNase-free water directly onto the membrane. Incubate for 1 min at room temperature. Centrifuge at 10,000 rpm for 1 min to elute. Take the flow-through, and reapply it to the membrane. Incubate for 1 min at room temperature, and centrifuge at 10,000 rpm for 1 min into the same collection tube. Add 0 or 10 μl of RNase-free water onto the membrane. Incubate for 1 min at room temperature (final elution volume: 1.5 μl/million BM and PBSC or 1 μl/million blood, minimal total volume: 12 μl). Centrifuge all the tubes at 14,000 rpm for 1 min to elute. Mix, quick spin, and record the total volume of each tube. Store the eluate at −80° C.
DNase Stock—Allow vial of 1500 Kunitz units DNase I, RNase-Free (lyophilized) to equilibrate to room temperature. Tap the vial on the table to get the powder down to the bottom of vial. Remove metal top and rubber cover. (Do not touch the inside of the rubber cover.) Add 550 μl of RNase-free water into vial and replace rubber cover. Dissolve powder in vial with water completely by gentle swirling, do not vortexing. DNase I is especially sensitive to physical denaturation. Aliquot stock solution into 1.5 mL sterile microfuge tubes and store at −20° C. for up to 9 months.

### Example 23

The purpose is for the cDNA synthesis with M-MLV Reverse Transcriptase from quantified total RNA. Moloney Murine Leukemia Virus Reverse Transcriptase (M-MLV RT) uses single-stranded RNA or DNA in the presence of a primer to synthesize a complementary DNA strand. This enzyme was isolated (1) from E. coli expressing a portion of the pol gene of M-MLV on a plasmid (2, 3). The enzyme is used to synthesize first-strand cDNA up to 7 KB.

Specimen—Frozen total RNA, minimum 2,500 ng in 9 μL.
Reagents—Nuclease-free water; Oligo(dT)₁₂₋₁₈ (500 μg/ml) primer (Invitrogen 18418-012); 10 mM dNTP Mix (Invitrogen 18427-013); M-MLV RT (200 units) (Invitrogen 28025-013); 5× First-Strand Buffer (provided with M-MLV RT); 1M DTT (provided with M-MLV RT); ABI probe sets of CHGA (Hs00154441_m1), DCX (Hs00167057_m1), DDC (Hs00168031_m1), and TH (Hs00165941_m1).
Equipment and Supplies—Pipettes and tips that are only used for RNA: 10 μl, 200 μl; Microcentrifuge (Eppendorf, model 5430R, Fisher Scientific, Cat. No. 05413804); 60 well 0.5 ml GeneAmp PCR System 9700 (Applied Biosystem, 4310899); Two Ice buckets; Timer.
Procedure—Label 0.5 mL microfuge tubes with Specimen ID #. Prepare a 1 μM Primer Mix [28 μL nuclease-free water+24 of each primer (18 μM stock)=36 μL] in a 0.5 mL tube labeled “mix”; mix by gentle vortexing; add 324 μL nuclease-free water to the same tube to make a final 0.1 μM Primer Mix; mix well and put on ice. Dilute RNA with nuclease-free water to make a total of 2,500 ng in 9 μL per tube. Add the following components to the microfuge tubes (either A or B):

### A—Individual Additions:

| | |
|---|---|
| Oligo(dT)₁₂₋₁₈ (500 μg/ml) primer | 1 μL |
| 10 mM dNTP Mix | 1 μL |
| Mixture of 0.1 μM of CHGA, DCX, DDC, and TH | 1 μL |

### B—Pooled Buffer 1:

For 24 samples, add the following in a 0.5 mL tube labeled PB1, mix and spin the PB1.

| | |
|---|---|
| Oligo(dT)₁₂₋₁₈ (500 μg/ml) primer | 25 μL |
| 10 mM dNTP Mix | 25 μL |
| Mixture of 0.1 μM of CHGA, DCX, DDC, and TH | 25 μL |

Add to each tube: PB1 (Pooled Buffer 1), 3 μL. Mix and quick spin. Place into ABI 9700. GeneAmp and run step 1: 65° C. for 6 min. Quickly chill on ice for 15 min. Quick spin. Add the following to each tube (either A or B):

### A: Individual Additions:

| | |
|---|---|
| 5 × First-Strand Buffer | 4 μL |
| 0.1M DTT | 2 μL |
| Nuclease-free water | 1 μL |

### B: Pooled Buffer 2:

For 24 samples, add the following in a 0.5 mL tube labeled PB2, mix and quick spin the PB2.

| | |
|---|---|
| 5 × First-Strand Buffer | 100 μL |
| 0.1M DTT | 50 μL |
| Nuclease-free water | 25 μL |

Add to each tube: PB2 (Pooled Buffer 2), 7 μL Mix and quick spin. Place into ABI 9700 GeneAmp and run step 2: 37° C. for 10 min. Samples are placed into the machine by batches of 16-20 samples with 5 minutes between each batch. Add the following: M-MLV RT (200 units), 1 μL. Mix by pipetting gently four times. Place into ABI 9700 GeneAmp and select step 3: 50 min at 45° C.; inactivate by heating at 70° C. for 15 min and cool to 4° C. Quick spin. Add 30 μL cold nuclease-free water to tube. The final concentration will be 2,500 ng/504 (50 ng/4). Mix and quick spin. Store the tubes at −20° C. freezer.

### REFERENCES

1. Hanahan D, Weinberg R A: Hallmarks of cancer: the next generation. Cell 144:646-74, 2011
2. Steidl C, Lee T, Shah S P, et al: Tumor-associated macrophages and survival in classic Hodgkin's lymphoma. N Engl J Med 362:875-85, 2010
3. Ueno T, Toi M, Saji H, et al: Significance of macrophage chemoattractant protein-1 in macrophage recruitment, angiogenesis, and survival in human breast cancer. Clin Cancer Res 6:3282-9, 2000
4. DeNardo D G, Berennan D J, Rexhepaj E, et al: Leukocyte complexity predicts breast cancer survival. Cancer Discovery 1:OF52-65, 2011
5. Bronkhorst I H, Ly L V, Jordanova E S, et al: Detection of M2-macrophages in uveal melanoma and relation with survival. Invest Ophthalmol Vis Sci 52:643-50, 2011
6. Kurahara H, Shinchi H, Mataki Y, et al: Significance of M2-polarized tumor-associated macrophage in pancreatic cancer. J Surg Res 167:e211-9, 2011
7. Cohn S L, Pearson A D, London W B, et al: The International Neuroblastoma Risk Group (INRG) classification system: an INRG Task Force report. J ClinOncol 27:289-97, 2009
8. Deyell R J, Attiyeh E F: Advances in the understanding of constitutional and somatic genomic alterations in neuroblastoma. Cancer Genet 204:113-21, 2011
9. Schmidt M L, Lukens J N, Seeger R C, et al: Biologic factors determine prognosis in infants with stage IV neuroblastoma: A prospective Children's Cancer Group study. J ClinOncol 18:1260-1268, 2000
10. Simon T, Hero B, Faldum A, et al: Long term outcome of high-risk neuroblastoma patients after immunotherapy with antibody ch14.18 or oral metronomic chemotherapy. BMC Cancer 11:21, 2011
11. Berthold F, Boos J, Burdach S, et al: Myeloablative megatherapy with autologous stem-cell rescue versus oral maintenance chemotherapy as consolidation treatment in patients with high-risk neuroblastoma: a randomised controlled trial. Lancet Oncol. 6:649-658, 2005
12. Cheung N K, Kushner B H, Kramer K: Monoclonal antibody-based therapy of neuroblastoma. HematolOncolClin North Am 15:853-66, 2001
13. Matthay K K, Reynolds C P, Seeger R C, et al: Long-term results for children with high-risk neuroblastoma treated on a randomized trial of myeloablative therapy followed by 13-cis-retinoic acid: a children's oncology group study. J ClinOncol 27:1007-13, 2009
14. Matthay K K, Villablanca J G, Seeger R C, et al: Treatment of high-risk neuroblastoma with intensive chemotherapy, radiotherapy, autologous bone marrow transplantation, and β-cis-retinoic acid. Children's Cancer Group. N. Engl. J. Med. 341:1165-1173, 1999
15. George R E, Li S, Medeiros-Nancarrow C, et al: High-risk neuroblastoma treated with tandem autologous peripheral-blood stem cell-supported transplantation: long-term survival update. J ClinOncol 24:2891-6, 2006
16. Yu A L, Gilman A L, Ozkaynak M F, et al: Anti-GD2 antibody with GM-CSF, interleukin-2, and isotretinoin for neuroblastoma. N Engl J Med 363:1324-34, 2010
17. Schmidt M L, lal A, Seeger R C, et al: Favorable prognosis for patients 12 to 18 months of age with stage 4 nonamplified MYCN neuroblastoma: a Children's Cancer Group Study. J. ClinOncol 23:6474-6480, 2005
18. Baker D L, Schmidt M L, Cohn S L, et al: Outcome after reduced chemotherapy for intermediate-risk neuroblastoma. N Engl J Med 363:1313-23, 2010
19. Asgharzadeh S, Pique-Regi R, Sposto R, et al: Prognostic significance of gene expression profiles of metastatic neuroblastomas lacking MYCN gene amplification. J. Natl. Cancer Inst. 98:1193-1203, 2006
20. Wei J S, Greer B T, Westermann F, et al: Prediction of clinical outcome using gene expression profiling and artificial neural networks for patients with neuroblastoma. Cancer Res. 64:6883-6891, 2004
21. Ohira M, Oba S, Nakamura Y, et al: Expression profiling using a tumor specific cDNA microarray predicts the prognosis of intermediate risk neuroblastomas. Cancer Cell 7:337-350, 2005
22. Kalbfleisch J, Prentice R: The statistical analysis of failure time data. New York, John Wiley and Sons, 1980
23. Mood A M, Garybill F A, Boes D C: Introduction to the theory of statistics. New York, McGraw-Hill, 1974
24. Simon R, Radmacher M D, Dobbin K, et al: Pitfalls in the use of DNA microarray data for diagnostic and prognostic classification. J. Natl. Cancer Inst. 95:14-18, 2003
25. Simon R: Development and evaluation of therapeutically relevant predictive classifiers using gene expression profiling. J. Natl. Cancer Inst. 98:1169-1171, 2006
26. Katoh M: Identification and characterization of FLJ10737 and CAMTA1 genes on the commonly deleted region of neuroblastoma at human chromosome 1p36.31-p36.23. Int. J. Oncol 23:1219-1224, 2003
27. Nakagawara A, Azar C G, Scavarda N J, et al: Expression and function of TRK-B and BDNF in human neuroblastomas. Mol. Cell Biol. 14:759-767, 1994
28. Nakagawara A, Arima-Nakagawara M, Scavarda N J, et al: Association between high levels of expression of the TRK gene and favorable outcome in human neuroblastoma. N. Engl. J. Med. 328:847-854, 1993
29. Henrich K O, Fischer M, Mertens D, et al: Reduced expression of CAMTA1 correlates with adverse outcome in neuroblastoma patients. Clin Cancer Res. 12:131-138, 2006
30. Biswas S K, Mantovani A: Macrophage plasticity and interaction with lymphocyte subsets: cancer as a paradigm. Nat Immunol 11:889-896, 2010
31. Leidi M, Gotti E, Bologna L, et al: M2 macrophages phagocytose rituximab-opsonized leukemic targets more efficiently than m1 cells in vitro. J Immunol 182:4415-22, 2009
32. Song L, Asgharzadeh S, Salo J, et al: Vα24-invariant NKT cells mediate antitumor activity via killing of tumor-associated macrophages. The Journal of Clinical Investigation 119:1524-1536, 2009
33. Ara T, Song L, Shimada H, et al: Interleukin-6 in the bone marrow microenvironment promotes the growth and survival of neuroblastoma cells. Cancer Res 69:329-37, 2009
34. Tan T T, Coussens L M: Humoral immunity, inflammation and cancer. CurrOpinImmunol 19:209-16, 2007
35. Bromberg J F, Wrzeszczynska M H, Devgan G, et al: Stat3 as an Oncogene. Cell 98:295-303, 1999
36. Klein B, Zhang X, Jourdan M, et al: Paracrine rather than autocrine regulation of myeloma-cell growth and differentiation by interleukin-6 Blood 73:517-526, 1989
37. de Visser K E, Korets L V, Coussens L M: De novo carcinogenesis promoted by chronic inflammation is B lymphocyte dependent. Cancer Cell 7:411-23, 2005
38. Andreu P, Johansson M, Affara N I, et al: FcR gamma activation regulates inflammation-associated squamous carcinogenesis. Cancer Cell 17:121-34
39. Suzuki T, Bogenmann E, Shimada H, et al: Lack of high-affinity nerve growth factor receptors in aggressive neuroblastomas. J. Natl. Cancer Inst. 85:377-384, 1993
40. Aoyama M, Asai K, Shishikura T, et al: Human neuroblastomas with unfavorable biologies express high levels of brain-derived neurotrophic factor mRNA and a variety of its variants. Cancer Lett. 164:51-60, 2001
41. Ohira M, Nakagawara A: Global genomic and RNA profiles for novel risk stratification of neuroblastoma. Cancer Science 101:2295-2301, 2010
42. Fischer M, Spitz R, Oberthür A, et al: Risk Estimation of Neuroblastoma Patients Using Molecular Markers. KlinPadiatr 220:137, 146, 2008
43. Attiyeh E F, London W B, Mossé Y P, et al: Chromosome 1p and 11q deletions and outcome in neuroblastoma. N Engl J Med 353:2243-53, 2005
44. Janoueix-Lerosey I, Schleiermacher G, Michels E, et al: Overall Genomic Pattern Is a Predictor of Outcome in Neuroblastoma. Journal of Clinical Oncology 27:1026-1033, 2009
45. Oberthuer A, Hero B, Berthold F, et al: Prognostic Impact of Gene Expression-Based Classification for Neuroblastoma. Journal of Clinical Oncology 28:3506-3515, 2010
46. De Preter K, Vermeulen J, Brors B, et al: Accurate Outcome Prediction in Neuroblastoma across Independent Data Sets Using a Multigene Signature. Clinical Cancer Research 16:1532-1541, 2010
47. Guo C, White P S, Hogarty M D, et al: Deletion of 11q23 is a frequent event in the evolution of MYCN single-copy high-risk neuroblastomas. Med. Pediatr. Oncol. 35:544-546, 2000
48. Plantaz D, Vandesompele J, Van Roy N, et al: Comparative genomic hybridization (CGH) analysis of stage 4 neuroblastoma reveals high frequency of 11 q deletion in tumors lacking MYCN amplification. International Journal of Cancer 91:680-686, 2001
49. Bagatell R, Rumcheva P, London W B, et al: Outcomes of children with intermediate-risk neuroblastoma after treatment stratified by MYCN status and tumor cell ploidy. Journal of Clinical Oncology 23:8819-8827, 2005
50. Bown N, Cotterill S, Lastowska M, et al: Gain of Chromosome Arm 17q and Adverse Outcome in Patients with Neuroblastoma. New England Journal of Medicine 340:1954-1961, 1999
51. Carén H, Kryh H, Nethander M, et al: High-risk neuroblastoma tumors with 11q-deletion display a poor prognostic, chromosome instability phenotype with later onset. Proceedings of the National Academy of Sciences 107:4323-4328, 2010
52. Guo X, Chen Q R, Song Y K, et al: Exon array analysis reveals neuroblastoma tumors have distinct alternative splicing patterns according to stage and MYCN amplification status. BMC Med Genomics 4:35, 2011
53. Teale J M: B cell immune repertoire diversifies in a predictable temporal order in vitro. J Immunol 135:954-8, 1985
54. Pabst H F, Kreth H W: Ontogeny of the immune response as a basis of childhood disease. J Pediatr 97:519-34, 1980
55. Smith D H, Peter G, Ingram D L, et al: Responses of children immunized with the capsular polysaccharide of Hemophilusinfluenzae, type b. Pediatrics 52:637-44, 1973
56. Parke J C, Jr., Schneerson R, Robbins J B, et al: Interim report of a controlled field trial of immunization with capsular polysaccharides of Haemophilusinfluenzae type b and group C Neisseria meningitidis in Mecklenburg county, North Carolina (March 1974-March 1976). J Infect Dis 136 Suppl:S51-6, 1977
57. Peltola H, Kayhty H, Sivonen A, et al: Haemophilusinfluenzae type b capsular polysaccharide vaccine in children: a double-blind field study of 100,000 vaccines 3 months to 5 years of age in Finland. Pediatrics 60:730-7, 1977
58. Pincus D J, Morrison D, Andrews C, et al: Age-related response to two Haemophilusinfluenzae type b vaccines. J Pediatr 100:197-201, 1982
59. Kayhty H, Karanko V, Peltola H, et al: Serum antibodies after vaccination with Haemophilusinfluenzae type b capsular polysaccharide and responses to reimmunization: no evidence of immunologic tolerance or memory. Pediatrics 74:857-65, 1984
60. Beiske K, Burchill S A, Cheung I Y, et al: Consensus criteria for sensitive detection of minimal neuroblastoma cells in bone marrow, blood and stem cell preparations by immunocytology and QRT-PCR: recommendations by the International Neuroblastoma Risk Group Task Force. Br J Cancer 100:1627-37, 2009
61. Naranjo A, Parisi M T, Shulkin B L, et al: Comparison of 123I-metaiodobenzylguanidine (MIBG) and 131I-MIBG semi-quantitative scores in predicting survival in patients with stage 4 neuroblastoma: a report from the Children's Oncology Group. Pediatr Blood Cancer 56:1041-5, 2011
62. Hodi F S, O'Day S J, McDermott D F, et al: Improved Survival with Ipilimumab in Patients with Metastatic Melanoma. New England Journal of Medicine 363:711-723, 2010
63. Beatty G L, Chiorean E G, Fishman M P, et al: CD40 Agonists Alter Tumor Stroma and Show Efficacy Against Pancreatic Carcinoma in Mice and Humans. Science 331:1612-1616, 2011
64. Brahmer J R, Drake C G, Wollner I, et al: Phase I Study of Single-Agent Anti-Programmed Death-1 (MDX-1106) in Refractory Solid Tumors: Safety, Clinical Activity, Pharmacodynamics, and Immunologic Correlates. Journal of Clinical Oncology 28:3167-3175, 2010
65. Asgharzadeh S, Pique-Regi R, Sposto R, et al: Prognostic significance of gene expression profiles of metastatic neuroblastomas lacking MYCN gene amplification. J. Natl. Cancer Inst. 98:1193-1203, 2006
66. Wei J S, Greer B T, Westermann F, et al: Prediction of clinical outcome using gene expression profiling and artificial neural networks for patients with neuroblastoma. Cancer Res. 64:6883-6891, 2004
67. Ohira M, Oba S, Nakamura Y, et al: Expression profiling using a tumor-specific cDNA microarray predicts the prognosis of intermediate risk neuroblastomas. Cancer Cell 7:337-350, 2005

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A process, comprising
providing a first composition comprising a plurality of isolated nucleic acids probes;
contacting the first composition to an RNA sample from a mammalian subject desiring a determination of the likelihood of neuroblastoma recurrence or response to chemotherapy, to produce one or more cDNA molecules;
providing a second composition comprising one or more isolated nucleic acids probes comprising a sequence capable of hybridizing to one or more nucleic acids selected from the group of genes consisting of PTPN5, GPATC4, H2AFV, FCGR3A; FCGR3B, CD14, PGM2L1, NTRK2, CD33, THAP2, IL6R, GFRA3, CAMTA1, IL10, and BTBD3;
contacting the second composition with the one or more cDNA molecules to amplify the one or more cDNA molecules; and
quantifying the expression level of the one or more genes to determine the likelihood of neuroblastoma recurrence or response to chemotherapy in the mammalian subject.

2. The process of claim 1, further comprising determining a progression score.

3. The process of claim 2, wherein the progression score is determined by the following formula:
$$B + {\sum }_{i = 1}^{14} {{c}_{i} ( Δ CT gene )}_{i}$$
wherein B is the intercept value, c is the coefficient of a gene, i is index of summation, and ΔCT is the change in cycle threshold value for the gene.

4. The process of claim 3, wherein the coefficient is selected from the group consisting of:

| Gene | Coefficient |
|---|---|
| PTPN5 | 0.72 |
| GPATC4 | 1.79 |
| H2AFV | −2.06 |
| FCGR3A; FCGR3B | 1.56 |
| CD14 | −2.52 |
| PGM2L1 | −1.36 |
| NTRK2 | −0.65 |
| CD33 | 1.28 |
| THAP2 | −1.60 |
| IL6R | 1.18 |
| GFRA3 | −0.43 |
| CAMTA1 | 0.71 |
| IL10 | −0.91 |
| BTBD3 | 0.85 |

5. The process of claim 3, wherein the intercept value is −3.38.

6. The process of claim 2, wherein the progression score is used to determine a course of therapy.

7. The process of claim 1, wherein the RNA sample is obtained from a tumor sample.

8. A process for determining the likelihood of neuroblastoma recurrence or response to chemotherapy, in a subject in need thereof, comprising:
providing a sample from the subject;
determining an expression level of genes selected from the group consisting of: PTPN5, GPATC4, H2AFV, FCGR3A; FCGR3B, CD14, PGM2L1, NTRK2, CD33, THAP2, IL6R, GFRA3, CAMTA1, IL10, and BTBD3, of the sample;
determining a progression score for the subject,
wherein a progression score of above a median risk score indicates a high likelihood of neuroblastoma recurrence or low likelihood of response to chemotherapy, a progression score below a median risk score indicates a low likelihood of neuroblastoma recurrence or a high likelihood of response to chemotherapy.

9. The process of claim 8, wherein the subject is a child with MYCN non-amplified metastatic neuroblastoma.

10. The process of claim 8, wherein the progression score is determined by the following formula:
$$B + {\sum }_{i = 1}^{14} {{c}_{i} ( Δ CT gene )}_{i}$$
wherein B is the intercept value, c is the coefficient of a gene, i is index of summation, and ΔCT is the change in cycle threshold value for the gene.

11. The process of claim 10, wherein the coefficient is selected from the group consisting of:

| Gene | Coefficient |
|---|---|
| PTPN5 | 0.72 |
| GPATC4 | 1.79 |
| H2AFV | −2.06 |
| FCGR3A; FCGR3B | 1.56 |
| CD14 | −2.52 |
| PGM2L1 | −1.36 |
| NTRK2 | −0.65 |
| CD33 | 1.28 |
| THAP2 | −1.60 |
| IL6R | 1.18 |
| GFRA3 | −0.43 |
| CAMTA1 | 0.71 |
| IL10 | −0.91 |
| BTBD3 | 0.85 |

12. The process of claim 10, wherein the intercept value is −3.38.

13. The process of claim 8, wherein the progression score is used to determine a course of therapy.

14. A process to detect a tumor cell, comprising:
providing a first composition comprising a plurality of isolated nucleic acids probes;
contacting the first composition to an RNA sample from a mammalian subject desiring a diagnosis or prognosis regarding a tumor to produce one or more cDNA molecules;
providing a second composition comprising isolated nucleic acids probes comprising a sequence capable of hybridizing to nucleic acids selected from the group of detection genes consisting of chromogranin A (“CHGA”), doublecortin (“DCX”), dopadecarboxylase (DDC), paired-like homeobox 2B (“PHOX2B”), and tyrosine hydroxylase (“TH”);
contacting the second composition with the one or more cDNA molecules to amplify the one or more cDNA molecules; and
quantifying the expression level of the one or more detection genes to detect a tumor cell.

15. The process of claim 14, further comprising determining a detection gene score from the expression level of the detection genes.

16. The process of claim 14, further comprising determining the absence of a tumor cell when the detection gene score is 40 or higher, and the low likelihood of disease progression.

17. The process of claim 14, further comprising determining the presence of a tumor cell when the detection gene score between 37 and 40, and a medium likelihood of disease progression.

18. The process of claim 14, further comprising determining the presence of a tumor cell when the detection gene score is less than 37, and a high likelihood of disease progression.

19. The process of claim 14, wherein the second composition further comprises one or
more isolated nucleic acids probes comprising a sequence capable of hybridizing to one or more nucleic acids selected from the group of housekeeping genes consisting of beta-2 microglobulin (“B2M”), glyceraldehyde-3-phosphate dehydrogenase (“GAPDH”), hypoxanthine guanine phosphoribosyl transferase (“HPRT1”), succinate dehydrogenase complex, subunit A (“SDHA”); and
the process further comprises quantifying the expression level of the one or more housekeeping genes.

20. The process of claim 14, wherein the second composition further comprises one or
more isolated nucleic acids probes comprising a sequence capable of hybridizing to one or more nucleic acids selected from the group of microenvironment genes consisting of CD14, CD16 (FCGR3B; 3A), CD163, CD19, CD34, CD4, CD40LG, CD86, CD8A, CSF1 (M-CSF), CSF1R (CD115), CTLA4, CX3CR, CXCL12, CXCR3, CXCR4, FLT1 (VEGFR1), FOXP3, GNLY, GZMB, HMOX1, IFNG, IL10, IL13, IL15, IL2RA, IL4, IL6, IL6R, IL7, IL7R, IL8, KDR (VEGFR2), KLRK1 (NKG2D), NCAM1, TBX21, TEK, TGFB1, and VEGFA; and
the process further comprises quantifying the expression level of the one or more microenvironment genes.

21. The process of claim 20, wherein the expression level of the one or more microenvironment genes provides information regarding the quality of a bone marrow sample.

22. The process of claim 14, wherein the RNA sample is obtained from mononuclear cells.

23. The process of claim 14, wherein the RNA sample is obtained from bone marrow cells, blood, or peripheral blood stem cell (“PBSC”).

24-38. (canceled)
